(19) United States
(12) Patent Application Publication
(10) Pub. No.: US 2024/0075025 A1
(43) Pub. Date: Mar. 7, 2024

(54) Therapy for Mitochondrial Fatty Acid Beta-Oxidation and Transport Disorders

(71) Applicant: University of Pittsburgh - Of the Commonwealth System of Higher Education, Pittsburgh, PA (US)

(72) Inventors: Al-Walid A. Mohsen, Gibsonia, PA (US); Gerard Vockley, Pittsburgh, PA (US)

(21) Appl. No.: 18/376,188

(22) Filed: Oct. 3, 2023

Related U.S. Application Data
(62) Division of application No. 17/344,084, filed on Jun. 10, 2021, now Pat. No. 11,813,258.
Application No. 17/344,084 is a division of application No. 16/345,757, filed on Apr. 29, 2019, now Pat. No. 11,077,105.
(60) Provisional application No. 62/422,124, filed on Nov. 15, 2016.

(51) Int. Cl. A61K 31/496; A61K 31/426; A61K 31/495; A61K 45/06; A61P 3/08

(52) U.S. Cl. CPC A61K 31/496; A61K 31/426; A61K 31/495; A61K 45/06; A61P 3/08; A61K 38/45

(57) ABSTRACT

Methods of treating mitochondrial fatty acid β-oxidation and/or transport disorders arising from mutant proteins in the mitochondrial fatty acid β-oxidation and transport metabolic pathways in patients are provided. The methods modulate the mitochondrial fatty acid β-oxidation pathway at the last step so that the product of the mutant protein accumulates and stabilizes the mutant protein and/or the substrate(s)/product(s) of the downstream reactions accumulate and possibly bind to allosteric sites on the mutant protein to stabilize it. Trimetazidine pharmacodynamics function as such in the β-oxidation pathway. Further, a synergistic effect is observed where a trimetazidine and PPARδ agonist combination enhanced enzyme activity and presence significantly more than either alone.

### CROSS-REFERENCE TO RELATED APPLICATIONS

This application is a divisional application of U.S. patent application Ser. No. 17/344,084, filed Jun. 10, 2021, which is a divisional application of U.S. patent application Ser. No. 16/345,757, filed Nov. 15, 2017, which is the United States national phase of International Application No. PCT/US2017/061712 filed Nov. 15, 2017, and claims priority to United States Provisional Patent Application No. 62/422,124 filed Nov. 15, 2016, the disclosure of each of which is hereby incorporated in its entirety by reference.

### STATEMENT REGARDING FEDERAL FUNDING

This invention was made with government support under Grant Nos. DK078775 and HD056004 awarded by the National Institutes of Health. The government has certain rights in the invention.

### SEQUENCE STATEMENT

The Sequence Listing associated with this application is filed in electronic format via Patent Center and is hereby incorporated by reference into the specification in its entirety. The name of the file containing the Sequence Listing is 2307098.xml. The size of the file is 9,329 bytes, and the file was created on Sep. 18, 2023.

### BACKGROUND

### Field of the Invention

Provided herein are methods and compositions for treatment of metabolic disorders, specifically to disorders relating to the β-oxidation pathway and/or fatty acid mitochondrial transport.

### Description of Related Art

Many genetic disorders, especially inborn errors of metabolism, are caused by the deficiency in the activity of one particular enzyme causing a biochemical pathway block. A biochemical pathway block causes the accumulation of the enzyme's substrate and the concomitant buildup of alternative metabolites that can be lethal or cause brain damage, within hours in some cases, and deficiency of an enzyme's pathway end product can also be life threatening. Genetic disorders that result from missense mutations resulting in the replacement of an amino acid residue with another causing reduction, or loss, of enzyme function can often render the mutated proteins structurally defective leading to their mis-folding and/or instability of folded mutants. In most cases, mutant enzymes that reach the tertiary and/or quaternary folded/assembled state may have partial activity, but they are usually thermolabile and vulnerable to proteolysis, making fever, strenuous exercise, or other stress factors life threatening decompensation triggers.

Often protein thermostability and vulnerability to proteolysis improve significantly by ligand binding. Ligands that improve protein stability include the protein or enzyme's own substrate, a substrate analog, or its reaction product(s). Other stabilizing ligands also include compositions that bind to allosteric sites, or small chaperone molecules that provide stability through mostly unknown mechanisms. Because the accumulation of an enzyme's substrate is large and often overwhelming, causing accumulation of toxic metabolites, management protocols of some diseases of inborn error of metabolism include omitting sources of substrates or prosubstrates of the deficient enzyme from the diet, if possible.

The acyl-coenzyme A (acyl-CoA) dehydrogenase (ACAD) family of enzymes are structurally and biochemically similar flavoenzymes consisting of 9 known members. Five of the ACADs; very long chain acyl-CoA dehydrogenase (VLCAD), acyl-CoA dehydrogenase 9 (ACAD9), long chain acyl-CoA dehydrogenase (LCAD), medium chain acyl-CoA dehydrogenase (MCAD), and short chain acyl-CoA dehydrogenase (SCAD), catalyze the first step of the spiral pathway with overlapping substrate chain length specificity, where each round shortens the carbon chain length by a 2-carbon unit in the form of acetyl-CoA as the pathway's main product (FIG. 1). Acetyl-CoA enters the tricarboxylic acid cycle for energy generation. The other four ACAD family members; isovaleryl-CoA dehydrogenase (IVD), isobutyryl-CoA dehydrogenase (IBD), short/branched chain (SBCAD), and glutaryl-CoA dehydrogenase (GD), function in the amino acid catabolism pathways.

Very long-chain acyl-CoA dehydrogenase deficiency (VLCADD) is an autosomal recessive metabolic genetic disorder of fatty acid metabolism. Its estimated frequency is at 1:30,000 to 1:100,000 births. Children with early-onset VLCADD present with symptoms within days or weeks after birth. These infants also show signs of hypoglycemia, irritability and lethargy. From ages two or three months to about two years, infants with this form of the disorder will be at risk for hypertrophic cardiomyopathy, abnormal heart rhythms and cardiorespiratory failure. Cardiomyopathy is rare in infancy, but may be life threatening when present. Later-onset VLCADD may present with recurrent episodes of lethargy and/or coma associated with hypoketotic hypoglycemia, during infancy, and hepatomegaly during childhood.

During later childhood and early adulthood, VLCADD patient hypoglycemia becomes less common and patients instead experience periodic attacks of muscle pain and rhabdomyolysis. Affected individuals may begin to experience recurrent metabolic acidosis, sudden respiratory arrest and even cardiac arrest. These symptoms may be associated with cardiomyopathy, lethargy, and coma. Without immediate treatment, such acute episodes may lead to potentially life-threatening complications. Individuals with VLCADD may have fat deposits and hepatomegaly, hypotonia, and/or evidence of cardiomyopathy. There may be hypertrophic or dilated cardiomyopathy. Significant increase in ROS in patients with VLCADD has also been found (unpublished data). Cardiomyopathy may lead to weakening in the force of heart contractions, decreased efficiency in the circulation of blood through the lungs and the rest of the body, and various associated symptoms that may depend upon the nature and severity of the condition, patient age, and other factors.

Medium acyl-CoA dehydrogenase (MCAD) catalyzes the α,β-dehydrogenation of C₆- to C₁₀-CoA ester substrates. MCAD deficiency is another fatty acid β-oxidation defect that rivals PKU in being the highest incidence among all metabolic genetic disorders. MCAD K304E is the most common (>90%) among all MCAD variants identified and is a thermally labile protein. Deficiency in the trifunctional protein (TFP) components' activities is another life threatening hereditary metabolic disorders caused by mutations in the HADHA or HADHB genes.

TFP is a hetero-octamer composed of four α- and four β-subunits, which the product from the VLCAD reaction, trans-2-enoyl-CoA, targets for the next three steps in the cycle. TFP catalyzes these three remaining steps of the β-oxidation cycle, with the α subunit (encoded by the HADHA gene) performing two functions, namely the hydration of trans-2-enoyl-CoA and the dehydrogenation of 3-hydroxyacyl CoA, and the β-subunit (encoded by the HADHB gene) performing the long-chain 3-ketoacyl-CoA thiolase (LCKAT) function. Defects in LCHAD and LCKAD, or TFP function, caused by mutations in the HADHA or HADHB genes, are life threatening hereditary metabolic disorders, with serious morbidity and mortality.

Current interventions for conditions caused by a mutation in an enzyme of the (β-oxidation pathway and/or fatty acid mitochondrial transport are extremely limited, and effective therapeutic interventions for such conditions are needed.

### SUMMARY

Provided herein is a method of treating a disorder caused by a mutation in any of the enzymes and/or proteins of the β-oxidation pathway and/or fatty acid mitochondrial transport in a patient in need thereof, comprising administering to the patient an effective amount of an inhibitor of activity of an enzyme of the β-oxidation pathway downstream in the β-oxidation pathway of the enzyme having the mutation, thereby increasing activity of the enzyme of the β-oxidation pathway of fatty acid mitochondrial transport having the mutation, wherein the product of the enzyme having the mutation stabilizes that unstable enzyme, or one or more substrates or products of the downstream enzyme binds allosterically to the mutant enzyme and stabilize that unstable enzyme.

Also provided herein is a method of increasing activity of a protein and/or enzyme of the β-oxidation pathway and/or fatty acid mitochondrial transporter protein/enzyme, the protein and/or enzyme having a mutation resulting in loss of its activity or stability, in a patient in need thereof, comprising administering to the patient an effective amount of an inhibitor of activity of an enzyme of the β-oxidation pathway downstream in the β-oxidation pathway of the enzyme having the mutation, thereby increasing activity of the upstream enzyme of the β-oxidation pathway and/or a fatty acid mitochondrial transporter protein/enzyme having a mutation, wherein the product of the enzyme having the mutation binds back and stabilizes that protein/enzyme.

### BRIEF DESCRIPTION OF THE DRAWINGS

The following drawings are for the purpose of illustration and description only and are not intended as a definition of the limit of the invention.

FIG. 1. The β-oxidation pathway. Five known enzymes catalyze the first step of the pathway with overlapping substrate chain length specificity. The rest of the pathway reactions are carried out by the Trifunctional Protein complex, TFP, which consists of four α and four β subunits carrying out three different functions.

FIGS. 2A-2F provide exemplary and non-limiting amino acid sequences for enzyme members of the human β-oxidation pathway, including: VLCAD (FIG. 2A), LCAD (FIG. 2B); MCAD (FIG. 2C); SCAD (FIG. 2D); TFP-α subunit (HADHA) (FIG. 2E); and TFP-β subunit (FIG. 2F).

FIG. 3. Effect of the presence of increasing amounts of TMZ for 48 hrs on VLCAD antigens' presence in FB833 and FB671 fibroblast cells as detected by western blot, A. Graph B, shows the observed changes normalized to GAPDH in the western blot, A, quantified using imageJ software. Graph B shows of the observed changes in the presence of VLCAD antigen normalized to DAPI detected in situ by immunostaining.

FIG. 4. Effect of the presence of increasing amounts of TMZ for 72 hrs on VLCAD and presence of TFP (combined α- and β-subunit) antigens' presence in FB834 fibroblast cells. Y axis represents fold change (ΔF) in relative VLCAD antigen present normalized to DAPI (for nuclear presence.)

FIG. 5. Effect of the presence of increasing amounts of TMZ for 48 hrs on VLCAD, MCAD, and LCAD antigens presence in FB834 fibroblast cells as detected by western blots. Y axis represents the fold change (ΔF) in protein presence.

FIG. 6. Effect of the presence of increasing amounts of TMZ for 72 hrs on VLCAD and TFP (combined α- and β-subunit) antigens' presence in FB671 fibroblast cells as detected by Immunostaining and quantification by ImageJ. Y axis represents the fold change (ΔF) in protein presence.

FIG. 7. Immunofluorescence staining for anti-MCAD (upper panel A & B) and anti-TFP (lower panel C & D) in MCAD deficient patient fibroblast FB831 in response to indicated TMZ treatment.

FIG. 8. Effect of the presence of increasing amounts, 2.5, 5, 10, and 20 μM, of TMZ in culture media for 72 hrs on MCAD (squares) and TFP antigen (circles) presence in FB831 MCAD deficient fibroblast cells. Y axis represents fold change (ΔF) in relative MCAD and TFP antigen present normalized to DAPI (for nuclear presence).

FIG. 9. Immunofluorescence staining for anti-HADHA, specific for the α-subunit or anti-HADHB, specific for the β-subunit, or anti-TFP, which detects both the α-subunit and β-subunit. in LCHAD deficient patient fibroblast Fb830 in response to indicated TMZ treatment.

FIG. 10. Effect of the presence of increasing amounts, 0.25, 0.5, 1.25, 2.5, 5.0 and 10 μM, of TMZ in culture media for 72 hrs on HADHA (circles) and HADHB antigen (triangles) presence in FB830 LCHAD deficient fibroblast cells. Y axis represents fold change (ΔF) in relative HADHA or HADHB antigen present normalized to DAPI (for nuclear presence).

FIG. 11. Effect of the presence of increasing amounts, 0.25, 0.5, 1.25, 2.5, 5.0 and 10 μM, of TMZ in culture media for 72 hrs on HADHA (circles), HADHB antigen (squares), and VLCAD (triangles) presence in FB842 LCKAT-deficient fibroblast cells with an HADHB S70P amino acid replacement. Y axis represents fold change (ΔF) in relative HADHA or HADHB antigen present normalized to DAPI (for nuclear presence).

FIG. 12. Effect of the presence of increasing amounts of TMZ for 48 hrs on VLCAD activity using C16-CoA as substrate, measured by the ETF fluorescence reduction assay in FB834 fibroblast cells. Y axis (ΔF) represents fold change in specific activity.

FIG. 13. Effect of the presence of increasing amounts of TMZ for 48 hrs on VLCAD activity using C16-CoA as substrate, measured by the ETF fluorescence reduction assay in FB671 fibroblast cells. Y axis (ΔF) represents fold change in specific activity.

FIG. 14. Effect of the presence of increasing amounts of TMZ, 0, 2.5, 5, and 10 μM on ACADVL expression in FB834 and TMZ at 0, 5, and 10 μM in FB671 compared to control (FB826) when incubated for 48 hr measured in terms of VLCAD RNA signal presence.

FIG. 15. Changes in the concentrations of acylcarnitine induced by the presence of TMZ at various concentrations for patient cell line FB834. Values shown for C16-carnitine are divided by 10.

FIG. 16. Changes in the concentrations of saturated long chain acylcarnitines induced by the presence of TMZ at various concentrations for patient cell line FB834. Values shown for C₁₆-carnitine are divided by 10.

FIG. 17. Changes in the concentrations of short chain acylcarnitine induced by the presence of TMZ at various concentrations for patient cell line FB834 (mutation unpublished). Values shown for acetylcarnitine and C₄-carnitine are divided by 100 and 10, respectively.

FIG. 18. Effect of various concentrations of TMZ on the presence of ROS as measured by MitoSOX Red in the VLCAD deficient cell line FB834.

FIG. 19. Effect of various concentrations of TMZ on the presence of ROS as measured by MitoSOX Red in the VLCAD deficient cell line FB671.

FIG. 20. Effect of various concentrations of GW501516, GW0742, and L-165,041 PAPRd agonists on VLCAD presence as observed on western blots (VLCAD (62 kDa)). Quantitation of the bands were performed using ImageJ software.

FIG. 21. Effect of various concentrations of GW501516 (GW16) on VLCAD enzyme activity in deficient cell line FB833 treated for 48 hrs using the ETF fluorescence reduction assay and C16-CoA as substrate at 30 μM. MCAD activity was measured using C8-CoA as substrate at 30 μM.

FIG. 22. Synergistic effect of trimetazidine and GW501516 (GW16) combination treatment of VLCAD deficient cell line FB833 for 48 hrs. Effect of treating with 1, 2.5, and 10 μM trimetazidine, 0.2 and 1 nM GW501516, and the combinations of TMZ and indicated on VLCAD enzyme activity in deficient cell line FB833. Enzyme activity was measured using the ETF fluorescence reduction assay and C16-CoA as substrate at 30 μM. All values are in fold change compared to no treatment normalized as 1 (100%).

FIG. 23. Effect of trimetazidine and GW501516 (GW16) combination treatment of VLCAD deficient cell line FB833 for 72 hrs on VLCAD presence detected by immunostaining microscopy. Shown is the effect of treating with 2.5 μM trimetazidine or 1 nM GW501516 (GW16), and the combinations of TMZ and indicated in figure. VLCAD antigen presence in situ was observed using cell immunostaining microscopy and quantitated using ImageJ software. All values are in fold change compared to no treatment normalized as 1 (100%).

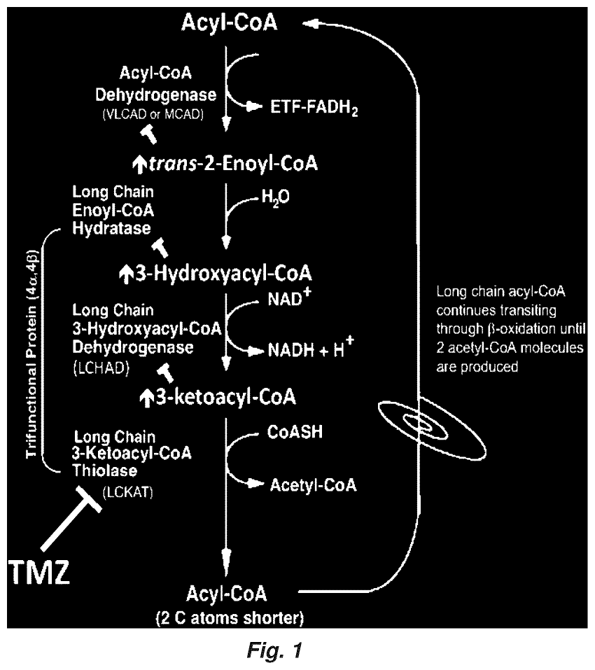

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions also refer to word forms, cognates and grammatical variants of those words or phrases.

As used herein, the terms “comprising,” “comprise” or “comprised,” and variations thereof, in reference to elements of an item, composition, apparatus, method, process, system, claim etc. are intended to be open-ended, meaning that the item, composition, apparatus, method, process, system, claim etc. includes those elements and other elements can be included and still fall within the scope/definition of the described item, composition, apparatus, method, process, system, claim etc. As used herein, “a” or “an” means one or more. As used herein “another” may mean at least a second or more.

As used herein, the terms “patient” or “subject” refer to members of the animal kingdom, including, but not limited to human beings.

As used herein, “alkyl” refers to straight, branched chain, or cyclic hydrocarbon (hydrocarbyl) groups including, for example, from 1 to about 20 carbon atoms, for example and without limitation C₁₋₃, C₁₋₆, C₁₋₁₀ groups, for example and without limitation, straight, branched chain alkyl groups such as methyl, ethyl, propyl, butyl, pentyl, hexyl, heptyl, octyl, nonyl, decyl, undecyl, dodecyl, and the like. “Substituted alkyl” refers to alkyl substituted at 1 or more, e.g., 1, 2, 3, 4, 5, or even 6 positions, which substituents are attached at any available atom to produce a stable compound, with substitution as described herein. “Optionally substituted alkyl” refers to alkyl or substituted alkyl. “Halogen,” “halide,” and “halo” refers to —F, —Cl, —Br, and/or —I. “Alkylene” and “substituted alkylene” refer to divalent alkyl and divalent substituted alkyl, respectively, including, without limitation, ethylene (—CH₂—CH₂—). “Optionally substituted alkylene” refers to alkylene or substituted alkylene.

“Aryl,” alone or in combination refers to an aromatic monocyclic or bicyclic ring system such as phenyl or naphthyl. “Aryl” also includes aromatic ring systems that are optionally fused with a cycloalkyl ring. A “substituted aryl” is an aryl that is independently substituted with one or more substituents attached at any available atom to produce a stable compound, wherein the substituents are as described herein. “Optionally substituted aryl” refers to aryl or substituted aryl. “Arylene” denotes divalent aryl, and “substituted arylene” refers to divalent substituted aryl. “Optionally substituted arylene” refers to arylene or substituted arylene.

“Heteroatom” refers to N, O, P and S. Compounds that contain N or S atoms can be optionally oxidized to the corresponding N-oxide, sulfoxide or sulfone compounds. “Hetero-substituted” refers to an organic compound in any embodiment described herein in which one or more carbon atoms are substituted with N, O, P or S.

“Substituted” or “substitution” refer to replacement of a hydrogen atom of a molecule or another atom or group with one or more different groups, such as halogen, alkyl, alkoxy, alkylthio, trifluoromethyl, acyloxy, hydroxy, mercapto, carboxy, aryloxy, aryl, arylalkyl, heteroaryl, amino, alkylamino, dialkylamino, morpholino, piperidino, pyrrolidin-1-yl, piperazin-1-yl, nitro, sulfato. Examples of substitutions are isosteric substitutions as described herein.

“Cycloalkyl” refer to monocyclic, bicyclic, tricyclic, or polycyclic, 3- to 14-membered ring systems, which are either saturated, unsaturated or aromatic. The cycloalkyl group may be attached via any atom. Cycloalkyl also contemplates fused rings wherein the cycloalkyl is fused to an aryl or hetroaryl ring. Representative examples of cycloalkyl include, but are not limited to cyclopropyl, cyclobutyl, cyclopentyl, and cyclohexyl. A cycloalkyl group can be unsubstituted or optionally substituted with one or more substituents as described herein. “Cycloalkylene” refers to divalent cycloalkyl. The term “optionally substituted cycloalkylene” refers to cycloalkylene that is substituted, e.g., with 1, 2 or 3 substituents, attached at any available atom to produce a stable compound, wherein the substituents are as described herein.

A “disorder caused by a mutation in an enzyme of the β-oxidation pathway and/or fatty acid mitochondrial transport,” is a condition, disease, defect, disorder, typically a congenital disorder or birth defect, that results from lowered activity of an enzyme of the β-oxidation pathway, such as an ACAD (VLCAD, LCAD, MCAD, or SCAD), the TFP, carnitine palmitoyltransferase I (CPT I), carnitine-acycarnitine translocase, and/or carnitine palmitoyltransferase II (CPT II). In aspects, the mutation is a missense mutation. The mutation results in an unstable or labile protein that is stabilized in the presence of and/or by a product(s)/substrate(s) of the enzyme. The stabilizing effect induced by binding of the product(s)/substrate(s) to the enzyme results in overall increase in enzyme activity, that is, the phenotypic effect of the enzyme, in a cell, e.g. of a patient. An enzyme substrate is the molecule upon, which the enzyme acts producing a product, which in the chemical sense can be a substrate since the reaction can run in reverse in some cases. The mutation typically is a missense mutation, such as arise from single nucleotide substitutions that changes the amino acid residue to one that disrupt the structural contribution of the normal residue to enzyme structure and/or function. The change can be a 3-nucleotide (or multiples of 3 to keep the protein code inframe) insertion or deletion, and the methods described herein are considered effective so long as activity of the mutant enzyme increases upon binding of the substrate in the enzyme assay described.

Provided herein are methods of stabilizing members of the β-oxidation pathway having mutations that are stabilized by ligand binding. In particular, provided herein are methods for treating a disorder caused by a mutation, for example and without limitation a missense mutation, in any of the enzymes/proteins of the β-oxidation pathway and/or fatty acid mitochondrial transport in a patient in need thereof.

Screening for small chaperone molecules that bind enzymes at sites away from their catalytic sites to stabilize structurally compromised mutants has been a major rational for drug development, stabilizing molecules, such as the enzyme's own reaction product or substrates/products of the downstream pathway steps that are already in situ would provide such stabilizing effect only if they are present at proper concentrations to bind to the defective enzyme/protein.

The above rational is the basis of the concept presented where enzymes that catalyze the pathway's downstream reactions to the defective mutant are targeted for inhibitory drug development with the aim of accumulating their substrate(s)/product(s) that may in turn bind to the pathway's upstream unstable variant enzyme (or subunit in a multi-enzyme complex). The downstream substrate(s)/product(s) may bind at the enzyme variant active site, or possibly allosteric site(s), to confer enough stability for improved function, thus alleviating the genetic disorder phenotype.

The protective effect conferred by ligand binding against proteolytic digestion has been a known phenomenon (Mohsen et al., 1995). The protective effect conferred by ligand binding against protein thermal unfolding has been demonstrated Nasser, I., et al. (2004). Thermal unfolding of medium-chain acyl-CoA dehydrogenase and iso(3)valeryl-CoA dehydrogenase: study of the effect of genetic defects on enzyme stability. Biochim Biophys Acta 1690, 22-32), effective methods of accomplishing that feat are not. For ACADs, in vitro thermal stability data with purified wild type recombinant human MCAD have shown that C₈-CoA binding induces 5-11° C. protection against thermal unfolding with the protection of the substrate analog 2-aza-C₈-CoA also occurring, but not as impressive (Nasser et al., 2004). Similarly, the effect of C₅-CoA binding on IVD, was in the 4-5° C. range. In the case of the two most common MCAD and IVD patient mutants, MCAD K304E and IVD A282V, the corresponding substrate/product provided thermally similar to the corresponding wild type. CoA persulfide, a substrate analog that binds tightly to certain ACADs, and confers protection to IVD against trypsin digestion. All members of the ACAD family of enzymes, TFP subunits, and mitochondrial transport proteins, can be safely presumed to behave similarly in the presence of their corresponding substrate/product. By stabilizing the mutant enzymes, diseases or conditions in patients resulting from deficiency of the mutant enzyme can be effectively treated. Thus, ACAD deficiencies and deficiencies in enzyme members of the β-oxidation pathway can be effectively treated by blocking, at least temporarily, LCKAT activity in a patient. When further combined with an activator of the β-oxidation pathway, such as a Peroxisome Proliferator-Activated Receptor delta (PPARδ) agonist, a β-oxidation pathway in a patient in which at least one enzyme member of the pathway is mutated, e.g., with a missense mutation, further enhanced activity of the mutant enzyme is observed.

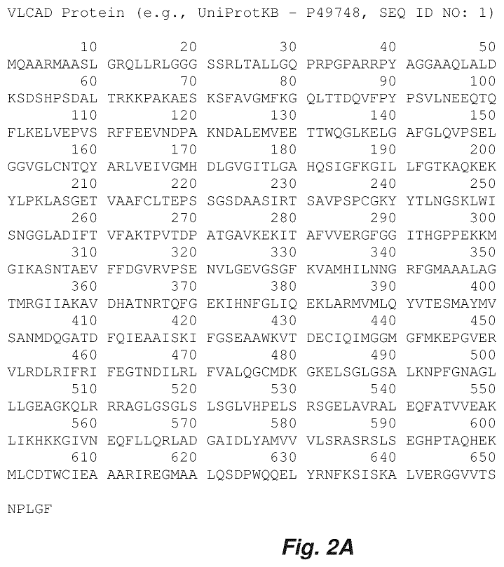

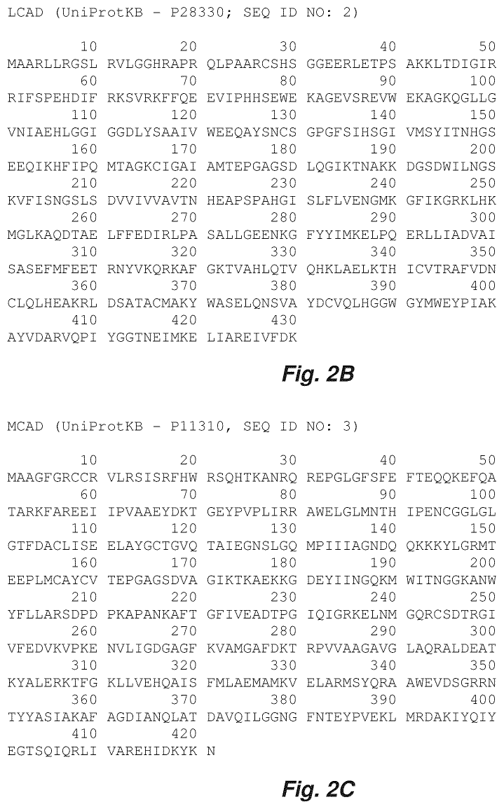

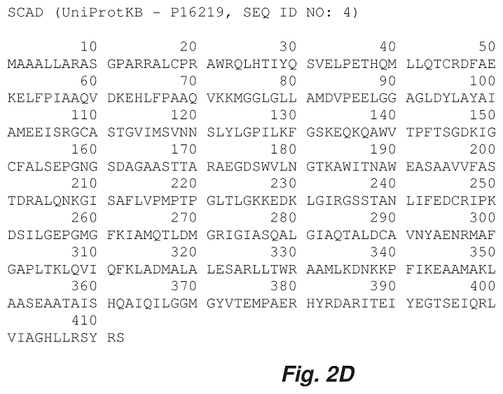

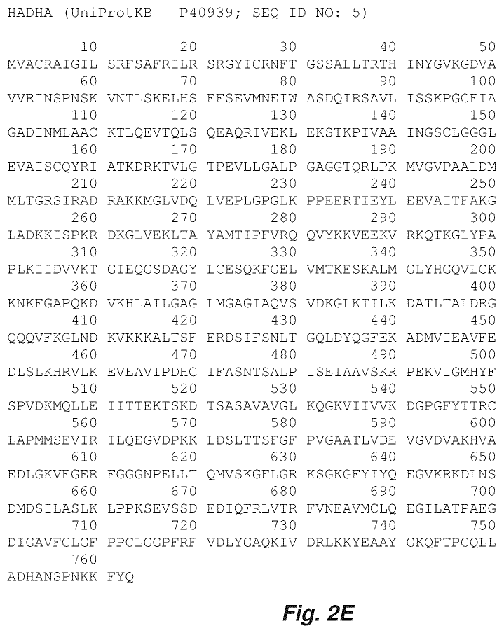

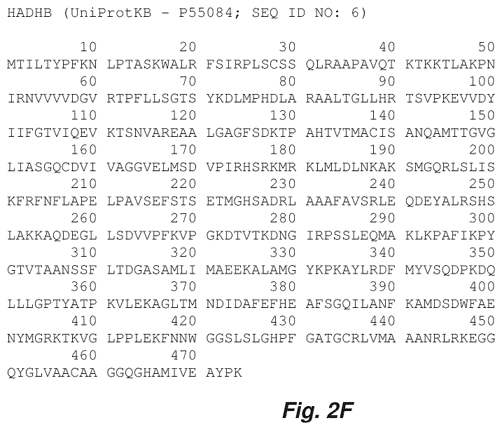

FIGS. 2A-2F provide exemplary amino acid sequences for enzyme members of the human β-oxidation pathway, including: VLCAD (example, FIG. 2A) and its isoenzymes, LCAD (FIG. 2B); MCAD (FIG. 2C); SCAD (FIG. 2D); TFP-α subunit (HADHA) (FIG. 2E); and TFP-β subunit (FIG. 2F). Missense mutations of those amino acid sequences are published. Exemplary missense mutations include more than 65 of VLCAD (See Table 1, Uniprot Accession No. D43682, Uniprot Accession No. P49748, and Pena et al., 2016), MCAD mutants including the well known K304E mutant (K329E, precursor numbering, shown in SEQ ID NO: 3) (Gregersen et al., 2008); E510Q of HADHA (or E474Q in reference to the mature HADHA protein).

TABLE 1

| | | Residue | |
|---|---|---|---|
| | | position | |
| | | (Precursor | |
| | | numbering) | |
| | | (VLCAD | |
| | | Accession: | Residue |
| | Nucleotide | P49748; | change |
| Publication | Mutation | see FIG. 2A) | (Reported) |
| Andresen et al., 1999 | 128G > A | | G3D |
| Watanabe, et al., 2000 | 194C > T | P65 | P65L |
| Zhang et al., 2014 | 215C > T | S72 | S72F |
| (Unpublished) | 439C > T | P147 | P147S |
| Andresen et al., 1999 | 473C > A | T118 | T118N |
| Andresen et al., 1999 | 476A > G | Q119 | Q119R |
| Laforet et al., 2009 | 455G > A | G152D | |
| Boneh et al., 2006 | 481G > A | A161T | A121T |
| (Vockley, Unpublished) | 482C > T | A161V | |
| Andresen et al., 1999 | 520G > A | V134 | V134M |
| Andresen et al., 1999 | 553G > A | G145 | G145S |
| (Vockley, Unpublished) | 622G > A | G208R | G168R |
| Mathur et al., 1999 | 637G > C | A213 | A173P |
| (Mohsen, Unpublished) | 520G > A | V174M | |
| Andresen et al., 1999 | 652G > A | E218 | E178K |
| Laforet et al., 2009 | 535G > T | | G179W |
| Andresen et al., 1999 | 728T > G | L243 | L203R |
| Mathur et al., 1999 | A739G | K247 | K207E |
| Andresen et al., 1999 | 740A > C | K247 | K207T |
| Zhang et al., 2014 | c.637G > A | A213 | A213T |
| Boneh et al., 2006 | 753-2A > C | | |
| Andresen et al., 1999; | 779C > T | T260 | T220M |
| Goetzman et al., 2007 | | | |
| Zhang et al., 2014 | 664G > C | G222 | G222R |
| (Vockley, Unpublished) | 689C > T | T230I | T230I |
| Watanabe et al., 2000 | 739A > C | | (K247Q) |
| | transversion | | |
| Laforet et al., 2009; | 842C > A | | A281D |
| Andresen et al., 1996A, 1999 | | | |
| (Vockley, Unpublished) | 848T > C | V283A | |
| (Vockley, Unpublished) | 865G > A | G289R | G289R |
| Andresen et al., 1999 | 869G > A | | G250D |
| Andresen et al., 1999 | 881G > A | | G254E |
| Andresen et al., 1999 | 897G > T | | K259N |
| (Vockley, Unpublished) | 898A > G | M300V | M300V |
| Andresen et al., 1999 | 950T > C | | V277A |
| | 956C > A | | |
| Andresen et al., 1999 | 1054A > G | | M312V |
| Andresen et al., 1999 | 1096C > T | | R326C |
| Antunes et al., 2013 | 1097G > A | R366 | R326H |
| Boneh et al., 2006; | | | |
| Andersen et al., 1999 | | | |
| Boneh et al., 2006 | 1117A > T | I373 | I333F |
| (Unpublished) | 1001T > G | M334R | M334R |
| (Unpublished) | 1066A > G | I356V | I356V |
| (Unpublished) | 1076C > T | A359V | A359V |
| Boneh et al., 2006 | 1153C > T | R385 | R345W |
| Andresen et al., 1999 | 1213G > C | | D365H |
| (Unpublished) | 1146G > C | K382N | K382N |
| Zhang et al., 2014 | 1310T > C | M437 | M437T |
| | | G439D | |
| Boneh et al., 2006; | 1322G > A | G441 | G401D |
| Manthur et al., 1999; | | | |
| Andersem et al., 1999 | | | |
| Andresen et al., 1999 | 1358G > A | | R413Q |
| Andresen et al., 1999 | 1360G > A | | D414N |
| Mathur et al., 1999 | 1372T > C | | F418L |
| | 1258A > C | I420L | |
| Andresen et al., 1999 | 1388G > A | | G423E |
| Andresen et al., 1999; | 1405C > T | | R429W |
| Goetzman et al., 2007 | | | |
| Andresen et al., 1999 | 1406G > A | | R429Q |
| Pons et al., 2000 | 1430G > A | | C437Y |
| Zhang et al., 2014; | 1349G > A | R450 | R450H |
| Boneh et al., 2006; | | | |
| Andersen et al., 1999 | | | |
| Andresen et al., 1999; | 1505T > C | | L462P |
| Goetzman et al., 2007 | | | |
| Zhang et al., 2014 | 1396G > T | D466 | D466Y |
| Laforet et al., 2009 | 1613G > C | | R538P |
| Mathur et al., 1999 | 1600G > A | | E454K |
| Aliefendio\check{g}lu et al., 2007; | 1367G > A | | R456H |
| Andresen et al., 1999 | | | |
| Pons et al, 2000; | 1375C > T | R459W | R459W |
| Andresen et al., 1999 | | | |
| Laforet et al., 2009 | 1376G > A | | R459Q |
| (Unpublished) | 1532G > A | R511Q | R511Q |
| (Vockley, Unpublished) | 1619T > C | L540P | |
| Andresen et al., 1999 | 1804C > A | | L562I |
| Mathur et al., 1999 | 1844G > A | | R575Q |
| (Mohsen, Unpublished) | 1825G > A | E609K | |
| Mathur et al., 1999 | 1844G > A | R615Q | R615Q |
| (Unpublished) | 1837C > G | R613G | R613G |

VLCAD is a homodimeric flavoenzyme and is the enzyme that catalyzes the first step in the fatty acid β-oxidation spiral where the long chain acyl-CoA substrate is converted to the trans-2-enoyl-CoA product. Specifically, VLCAD catalyzes the α,β-dehydrogenation of C₁₄-C₂₂-CoA ester substrates. VLCAD deficiency is an inborn error of metabolism caused by one homozygous mutation, or two (different one in each allele, example FB833, V174M and E609K used in this study) heterozygous, of a high number of mutations that has been identified and essentially cause life threatening decrease in mitochondrial long chain fatty acid β-oxidation in severe cases. Among the mutations identified in the VLCAD gene, more than 65 have been missense mutations that cause different degrees of instabilities, with VLCAD A283V mutant being more common than others (See Table 1 and Pena et al., 2016). Since VLCAD is the entry enzyme into the pathway, complete deficiency of VLCAD essentially shuts down most of the long chain fatty acid mitochondrial-oxidation, an essential energy source and provider of the medium chain acyl-CoAs that continue to complete the conversion of the fatty acid to the acetyl-CoA end product. VLCAD is most active when C₁₆-CoA is used as the substrate, which is converted to trans-2-enoyl-CoA, which is released upon the transfer of electrons to the second substrate, the electron transfer flavoprotein (ETF). The trans-2-enoyl-CoA product can still bind to the oxidized VLCAD inhibiting the reaction and so must be cleared by the next step in the pathway, which is catalyzed by the long chain enoyl-CoA hydratase. This latter enzyme, however, is part of the Trifunctional Protein (TFP) complex, which consists of 4 α-subunits and 4 β-subunits. The TFP also includes 3-hydroxyacyl-CoA dehydrogenase and the long chain 3-ketoacyl-CoA thiolase. Since substrate channeling to the various active sites within the complex is expected, inhibiting any of the activities of the complex components is likely to negatively affect the other two.

Trimetazidine (TMZ, 1-(2,3,4-trimethoxybenzyl)piperazine, PubChem CID: 21109; molar mass 266.336 g/mol):

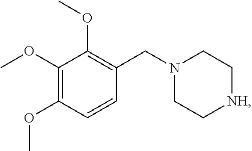 [SMILES: COc1ccc(CN2CCNCC2)c(OC)c1OC]

is an inhibitor of the downstream “last” reaction in the β-oxidation cycle catalyzed by LCKAT. Here, TMZ is used to bind to/inhibit LCKAT activity leading to the stabilization of LCKAT itself, and the upstream β-oxidation cycle components including, LCAHD, VLCAD, and MCAD, and it is expected to stabilize other members of the TFP complex(es) and mitocondrial fatty acid transport proteins, and so can be used to treat deficiencies of the mitochondrial β-oxidation cycle and also deficiencies in mitochondrial membrane fatty acid transport proteins, e.g., Carnitine palmitoyltransferase I (CPT I) and Carnitine palmitoyltransferase 2 (CPT II). TMZ is approved by the FDA in the USA and elsewhere for use in angina pectoris due to ischemia of the heart muscle. Its generic version is manufactured globally and is available in 90 countries. Compared to conventional therapy, TMZ is reported to have shown significant improvements in non-ischemic and ischemic cardiomyopathy. The mechanism of the TMZ effect results from the shift of cardiac energy fulfillment from fatty acid oxidation to glucose oxidation. Since ˜80% of cardiac cells rely on fatty acid as a source of energy, by shifting reliance of cardiac cells for energy from fatty acids to glucose, oxygen demand is decreased.

TMZ derivatives having the ability to inhibit LCKAT activity also may be used to inhibit LCKAT activity, e.g. in a cell or a patient, thereby having the same effect as TMZ in causing accumulation of a substrate of an upstream mutated enzyme, thereby stabilizing the enzyme. TMZ and derivatives thereof are disclosed in U.S. Pat. No. 5,283,246, incorporated herein by reference. Non-limiting examples of other potentially useful compounds with the TMZ scaffold that may offer advantage(s) over TMZ include TMZ-NH (4-(2,2,5,5-tetramethylpyrrolinyl-3)-1-(2,3,4-trimethoxybenzyl)piperazine) and TMZ-ΦNH (4-(2,2,5,5-tetramethyl-4-phenylpyrrolinyl-3-)-1-(2,3,4-trimethoxybenzyl) piperazine, see below), as they have shown anti-ROS activity and other improved parameters in ischemic heart disease parameters that are pertinent to the application herein. TMZ derivatives include 4-substituted piperazine TMZ derivatives (structure 1),

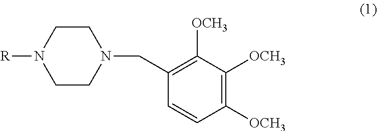

where R is, for example, a 5 to 7 member hydrocarbon or heterocyclic group substituted with from 1 to 3 N, O, and/or S hetero-atoms, and in one example is saturated, and in another is unsaturated or aromatic, and in a further example comprises from 1 to 3 N atoms, for example a pyrrolinyl group or a substituted pyrollinyl group, substituted with a C₁-C₆ hydrocarbon, such as a phenyl or a C₁-C₆ alkyl group, such as 2,2,5,5-tetramethylpyrrolinyl group (TMZ-NH):

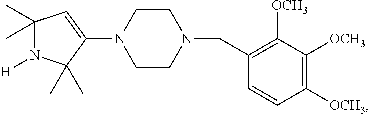 [SMILES: COc1ccc(CN2CCN(C3=CC(C)(C)NC3(C)C)CC2)c(OC)c1OC]

or 2,2,5,5-tetramethyl-4-phenylpyrrolinyl group, (TMZ-ΦNH):

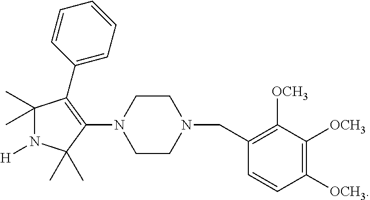 [SMILES: COc1ccc(CN2CCN(C3=C(c4ccccc4)C(C)(C)NC3(C)C)CC2)c(OC)c1OC]

Exemplary TMZ derivatives are described in Kutala, V. K., et al. (2006) (Attenuation of myocardial ischemia-reperfusion injury by trimetazidine derivatives functionalized with antioxidant properties. J Pharmacol Exper Therapeut 317 (3):921-928); Ancerewicz, J., et al. (1998) (Structure—property relationships of trimetazidine derivatives and model compounds as potential antioxidants. Free Rad. Biol Med 25 (1):113-120); and Kálai, T., et al. (2006) (Structure—activity studies on the protection of Trimetazidine derivatives modified with nitroxides and their precursors from myocardial ischemia-reperfusion injury. Bioorg Medicinal Chem 14:5510-5516). TMZ derivatives include isosteres of TMZ, in which one or more group of structure 1, above, is substituted with a similar group, resulting in a bioisostere molecule with the same overall therapeutic effect, e.g., inhibition of LCKAT, but potentially with attenuated toxicity, activity and/or pharmacokinetics. Non-limiting, and exemplary isosteric substitutions include, as groups: H, deuterium, and F; Me, NH₂, OH, F, and Cl; CI, PH₂, SH, and CN; Br, isopropyl, and CF₃; I, tert-butyl, and CF₃; CH₂, NH, O, and S; CO₂R, CONHR, COSR, and COCH₂R; —CH═, —N═, and —S—; C and Si; phenyl, pyridyl, thiophene, and 4-fluorophenyl; —OH, —F, and —OMe; and —O— and —CF₂—. Additional isosteric substitutions are known and can result in a compound with similar activity to TMZ.

Provided herein according to one aspect of the invention is a method of treating a disorder caused by a mutation, e.g., a missense mutation, in an enzyme of the β-oxidation pathway or fatty acid transport into mitochondria, in a patient in need thereof, comprising inhibiting activity of an enzyme of the β-oxidation pathway downstream in the β-oxidation pathway to the enzyme having the missense mutation, thereby increasing activity of the an enzyme of the β-oxidation pathway or fatty acid transport into mitochondria having the missense mutation. In one aspect, the activity of an enzyme of the β-oxidation pathway is inhibited by TMZ, a derivative thereof, a pharmaceutically acceptable salt thereof, or a pharmaceutically-acceptable ester thereof. In another aspect, the enzyme of the β-oxidation pathway downstream in the β-oxidation pathway to the enzyme having the missense mutation is the TFP subunit LCKAT (encoded by HADHB).

For therapeutic use, salts of the compounds are those wherein the counter-ion is pharmaceutically acceptable (pharmaceutically-acceptable salts). However, salts of acids and bases which are non-pharmaceutically acceptable may also find use, for example, in the preparation or purification of a pharmaceutically acceptable compound.

Pharmaceutically acceptable salts as mentioned herein are meant to comprise the therapeutically active non-toxic acid and base addition salt forms which the compounds are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids. Conversely the salt forms can be converted by treatment with an appropriate base into the free base form.

The compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like. The term “addition salt” as used hereinabove also comprises the solvates which the compounds described herein are able to form. Such solvates are for example hydrates, alcoholates and the like.

The term “quaternary amine” as used hereinbefore defines the quaternary ammonium salts which the compounds are able to form by reaction between a basic nitrogen of a compound and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen.

Pharmaceutically acceptable counterions, such as, without limitation, chloro, bromo, iodo, trifluoroacetate, and acetate can be introduced using ion exchange resins.

“Pharmaceutically acceptable esters” includes those derived from compounds described herein that are modified to include a carboxyl group. An in vivo hydrolysable ester is an ester, which is hydrolyzed in the human or animal body to produce the parent acid or alcohol. Representative esters thus include carboxylic acid esters in which the non-carbonyl moiety of the carboxylic acid portion of the ester grouping is selected from straight or branched chain alkyl (for example, methyl, n-propyl, t-butyl, or n-butyl), cycloalkyl, alkoxyalkyl (for example, methoxymethyl), aralkyl (for example benzyl), aryloxyalkyl (for example, phenoxymethyl), aryl (for example, phenyl, optionally substituted by, for example, halogen, C₁₋₄ alkyl, or C₁₋₄ alkoxy) or amino); sulphonate esters, such as alkyl- or aralkylsulphonyl (for example, methanesulphonyl); or amino acid esters (for example, L-valyl or L-isoleucyl). A “pharmaceutically acceptable ester” also includes inorganic esters such as mono-, di-, or tri-phosphate esters. In such esters, unless otherwise specified, any alkyl moiety may include from 1 to 18 carbon atoms, such as from 1 to 6 carbon atoms, or from 1 to 4 carbon atoms. Any cycloalkyl moiety present in such esters advantageously contains from 3 to 6 carbon atoms. Any aryl moiety present in such esters advantageously comprises a phenyl group that is optionally substituted. Pharmaceutically acceptable esters thus include C₁-C₂₂ fatty acid esters, such as acetyl, t-butyl or long chain straight or branched unsaturated or omega-6 monounsaturated fatty acids such as palmoyl, stearoyl and the like. Alternative aryl or heteroaryl esters include benzoyl, pyridylmethyloyl and the like any of which may be substituted. Additional pharmaceutically acceptable esters include aliphatic L-amino acid esters such as leucyl, isoleucyl and valyl.

Prodrugs of the disclosed compounds also are contemplated herein. A prodrug is an active or inactive compound that is modified chemically through in vivo physiological action, such as hydrolysis, metabolism and the like, into an active compound following administration of the prodrug to a subject. The term “prodrug” as used throughout this text means the pharmacologically acceptable derivatives such as esters, amides and phosphates, such that the resulting in vivo biotransformation product of the derivative is the active drug as defined in the compounds described herein. Prodrugs preferably have excellent aqueous solubility, increased bioavailability and are readily metabolized into the active inhibitors in vivo. Prodrugs of a compounds described herein may be prepared by modifying functional groups present in the compound in such a way that the modifications are cleaved, either by routine manipulation or in vivo, to the parent compound. The suitability and techniques involved in making and using prodrugs are well known by those skilled in the art.

Protected derivatives of the disclosed compounds also are contemplated. Many suitable protecting groups for use with the disclosed compounds are broadly-known in the art. In general, protecting groups are removed under conditions which will not affect the remaining portion of the molecule. These methods are well known in the art and include acid hydrolysis, hydrogenolysis and the like. One method involves the removal of an ester, such as cleavage of a phosphonate ester using Lewis acidic conditions, such as in TMS-Br mediated ester cleavage to yield the free phosphonate. A second method involves removal of a protecting group, such as removal of a benzyl group by hydrogenolysis utilizing palladium on carbon in a suitable solvent system such as an alcohol, acetic acid, and the like or mixtures thereof. A t-butoxy-based group, including t-butoxy carbonyl protecting groups can be removed utilizing an inorganic or organic acid, such as HCl or trifluoroacetic acid, in a suitable solvent system, such as water, dioxane and/or methylene chloride. Another exemplary protecting group, suitable for protecting amino and hydroxy functions amino is trityl. Other conventional protecting groups are known and suitable protecting groups can be selected by those of skill in the art in consultation with any of the large number of broadly-available publications. When an amine is deprotected, the resulting salt can readily be neutralized to yield the free amine. Similarly, when an acid moiety, such as a phosphonic acid moiety is unveiled, the compound may be isolated as the acid compound or as a salt thereof.

According to one aspect, amine side chains are protected using protective groups, for example and without limitation by acylation. Protecting groups are known in the art and include, without limitation: 9-fluorenylmethyloxy carbonyl (Fmoc), t-butyloxycarbonyl (Boc), benzhydryloxycarbonyl (Bhoc), benzyloxycarbonyl (Cbz), O-nitroveratryloxycarbonyl (Nvoc), benzyl (Bn), allyloxycarbonyl (alloc), trityl (Trt), I-(4,4-dimethyl-2,6-dioxacyclohexylidene)ethyl (Dde), diathiasuccinoyl (Dts), benzothiazole-2-sulfonyl (Bts), dimethoxytrityl (DMT) and monomethoxytrityl (MMT) groups. A protecting group also includes acyl groups, such as acetyl groups, for example, as described.

Unless indicated otherwise, for instance in a structure, all compounds and/or structures described herein comprise all possible stereoisomers, individually or mixtures thereof. The compound and/or structure may be an enantiopure preparation consisting essentially of an (−) or (+) enantiomer of the compound, or may be a mixture of enantiomers in either equal (racemic) or unequal proportions.

The compounds typically are administered in an amount and dosage regimen to treat a condition or symptom of a condition in a patient caused by a missense mutation in an enzyme of the β-oxidation pathway. The compounds may be administered in any manner that is effective to treat, mitigate or prevent a condition or symptom of a condition in a patient caused by a missense mutation in an enzyme of the β-oxidation pathway. Examples of delivery routes include, without limitation: topical, for example, epicutaneous, inhalational, enema, ocular, otic and intranasal delivery; enteral, for example, orally, by gastric feeding tube, or rectally; and parenteral, such as, intravenous, intraarterial, intramuscular, intracardiac, subcutaneous, intraosseous, intradermal, intrathecal, intraperitoneal, transdermal, iontophoretic, transmucosal, epidural and intravitreal, with oral, intravenous, intramuscular and transdermal approaches being preferred in many instances.

As indicated above, and in the examples, a useful dosage of the inhibitor of the activity of an enzyme of the β-oxidation pathway ranges from, for example 10 μg to 10 mg/Kg body weight/day, and in one aspect ranges from 0.05 mg to 0.5 mg/Kg body weight/day. Different drug products will have different specific activities and therefore dosage is optimized for any particular drug product. Of note, the exemplary dosage range for TMZ ranges from 0.05 mg to 0.5 mg/Kg body weight/day, which is lower that a typical dosage of that compound for treatment of ischemic heart disease. An “effective amount” of the compound or composition described herein is an amount effective in a dosage regimen (amount of the compound and timing and mode of delivery), to achieve a desired end-point, such as maintaining concentrations at a site of treatment within a range effective to achieve an outcome. Suitable outcomes include overcoming a metabolic block in a patient caused by a missense mutation in an enzyme of the β-oxidation pathway, or improvement of one or more symptoms of a condition caused by a missense mutation in an enzyme of the β-oxidation pathway, for example as described herein. In aspects, the compositions can be given to a patient in a pulsed fashion in which an amount of the compound is provided that is effective to cause a partial or complete block in the β-oxidation pathway, followed by a time period in which the amount of the compound is reduced such that the block, e.g. of the 3-ketoacyl-CoA thiolase of the TFP, is released, permitting the β-oxidation pathway to proceed to its end (where 3-ketoacyl-CoA is converted to acyl-CoA by the 3-ketoacyl-CoA thiolase activity of the TFP). The amount of the compound administered to a patient, and the timing of the block, and release of the block, can be adjusted to an individual patient and/or to the specific mutation present in a patient.

The compounds may be compounded or otherwise manufactured into a suitable composition for use, such as a pharmaceutical dosage form or drug product in which the compound is an active ingredient. Compositions may comprise a pharmaceutically acceptable carrier, or excipient. An excipient is an inactive substance used as a carrier for the active ingredients of a medication. Although “inactive,” excipients may facilitate and aid in increasing the delivery or bioavailability of an active ingredient in a drug product. Non-limiting examples of useful excipients include: antiadherents, binders, rheology modifiers, coatings, disintegrants, emulsifiers, oils, buffers, salts, acids, bases, fillers, diluents, solvents, flavors, colorants, glidants, lubricants, preservatives, antioxidants, sorbents, vitamins, sweeteners, etc., as are available in the pharmaceutical/compounding arts.

Useful dosage forms include: intravenous, intramuscular, or intraperitoneal solutions, oral tablets or liquids, topical ointments or creams and transdermal devices (e.g., patches). In one embodiment, the compound is a sterile solution comprising the active ingredient (drug, or compound), and a solvent, such as water, saline, lactated Ringer's solution, or phosphate-buffered saline (PBS). Additional excipients, such as polyethylene glycol, emulsifiers, salts and buffers may be included in the solution.

Therapeutic/pharmaceutical compositions are prepared in accordance with acceptable pharmaceutical procedures, such as described in Remington: The Science and Practice of Pharmacy, 21st edition, ed. Paul Beringer et al., Lippincott, Williams & Wilkins, Baltimore, MD Easton, Pa. (2005) (see, e.g., Chapters 37, 39, 41, 42 and 45 for examples of powder, liquid, parenteral, intravenous and oral solid formulations and methods of making such formulations).

In aspects, the compounds or compositions are co-administered with one or more additional therapeutic agents. In one aspect, the additional therapeutic agent is a Peroxisome Proliferator-Activated Receptor delta (PPARδ) agonist; that is, a drug that acts on PPARδ to modulateor increase, PPARδ activity. In one example, the PPARδ agonist is GW 501516 (2-[2-methyl-4-[[4-methyl-2-[4-(trifluoromethyl)phenyl]-1,3-thiazol-5-yl]methylsulfanyl]phenoxy]acetic acid), having the structure:

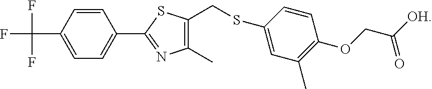 [SMILES: Cc1cc(SCc2sc(-c3ccc(C(F)(F)F)cc3)nc2C)ccc1OCC(=O)O]

Certain PPARδ agonists are available commercially from Mitobridge of Cambridge, Massachusetts. Further, United States Patent Application Publication Nos. 20160023991, 20170226154, 20170304255, and 20170305894, incorporated herein by reference for their disclosure of PPARδ agonists, disclose a large number of PPARδ agonists, including, without limitation:
6-(2-((N-isopropyl-[1,1′-biphenyl]-4-carboxamido)methyl)phenoxy)hexanoic acid; ethyl 6-(2-(1-(4-bromo-N-cyclopropylbenzamido)-2-(tert-butylamino)-2-oxoethyl)phenoxy)hexanoate; ethyl 6-(2-(2-(tert-butylamino)-1-(N-cyclopropyl-[1,1′-biphenyl]-4-carboxamido)-2-oxoethyl)phenoxy)hexanoate; ethyl 6-(2-(2-amino-1-(N-cyclopropyl-[1,1′-biphenyl]-4-carboxamido)-2-oxoethyl)phenoxy)hexanoate; 6-(2-(2-amino-1-(N-cyclopropyl-[1,1′-biphenyl]-4-carboxamido)-2-oxoethyl)phenoxy)hexanoic acid; 6-(2-(2-(tert-butylamino)-1-(N-cyclopropyl-[1,1′-biphenyl]-4-carboxamido)-2-oxoethyl)phenoxy)hexanoic acid; 6-(2-((N-cyclopropyl-[1,1′-biphenyl]-4-carboxamido)methyl)phenoxy)hexanoic acid; N-(2-amino-1-(2-((6-(hydroxyamino)-6-oxohexyl)oxy)phenyl)-2-oxoethyl)-N-cyclopropyl-[1,1′-biphenyl]-4-carboxamide; 6-(2-((N-cyclopropyl-4-(pyridin-4-yl)benzamido)methyl)phenoxy)hexanoic acid; 6-(2-((N-cyclopropyl-4-(pyridin-3-yl)benzamido)methyl)phenoxy)hexanoic acid; 6-(2-((N-cyclopropyl-4-(1H-pyrazol-4-yl)benzamido)methyl)phenoxy)hexanoic acid; 6-(2-((N-cyclopropyl-4-(furan-2-yl)benzamido)methyl)phenoxy)hexanoic acid; 6-(2-((N-cyclopropyl-4-(furan-3-yl)benzamido)methyl)phenoxy)hexanoic acid; 6-(2-((N-cyclopropyl-4-(thiophen-2-yl)benzamido)methyl)phenoxy)hexanoic acid; 6-(2-((N-cyclopropyl-4-(thiophen-3-yl)benzamido)methyl)phenoxy)hexanoic acid; 6-(2-((N-benzyl-[1,1′-biphenyl]-4-carboxamido)methyl)phenoxy)hexanoic acid; 6-(2-((N-benzyl-4-(pyridin-3-yl)benzamido)methyl)phenoxy)hexanoic acid; 6-(2-((N-benzyl-4-(pyridin-4-yl)benzamido)methyl)phenoxy)hexanoic acid; 6-(2-((N-benzyl-4-(1H-pyrazol-4-yl)benzamido)methyl)phenoxy)hexanoic acid; 6-(2-((N-benzyl-4-(thiophen-2-yl)benzamido)methyl)phenoxy)hexanoic acid; 6-(2-((N-benzyl-4-(furan-3-yl)benzamido)methyl)phenoxy)hexanoic acid; 6-(2-((N-benzyl-4-(furan-2-yl)benzamido)methyl)phenoxy)hexanoic acid; 6-(2-((N-benzyl-4-(thiophen-3-yl)benzamido)methyl)phenoxy)hexanoic acid; N-benzyl-N-(2-((6-(hydroxyamino)-6-oxohexyl)oxy)benzyl)-4-(pyridin-4-yl)benzamide; N-benzyl-N-(2-((6-(hydroxyamino)-6-oxohexyl)oxy)benzyl)-[1,1′-biphenyl]-4-carboxamide; N-benzyl-N-(2-((6-(hydroxyamino)-6-oxohexyl)oxy)benzyl)-4-(pyridin-3-yl)benzamide; N-benzyl-N-(2-((6-(hydroxyamino)-6-oxohexyl)oxy)benzyl)-4-(1H-pyrazol-4-yl)benzamide; N-benzyl-N-(2-((6-(hydroxyamino)-6-oxohexyl)oxy)benzyl)-4-(thiophen-2-yl)benzamide; N-benzyl-4-(furan-2-yl)-N-(2-((6-(hydroxyamino)-6-oxohexyl)oxy)benzyl)benzamide; N-benzyl-4-(furan-3-yl)-N-(2-((6-(hydroxyamino)-6-oxohexyl)oxy)benzyl)benzamide; 6-(2-((N-(sec-butyl)-[1,1′-biphenyl]-4-carboxamido)methyl)phenoxy)hexanoic acid; N-benzyl-N-(2-((6-(hydroxyamino)-6-oxohexyl)oxy)benzyl)-4-(thiophen-3-yl)benzamide; 6-(2-((N-(sec-butyl)-4-(pyridin-3-yl)benzamido)methyl)phenoxy)hexanoic acid; 6-(2-((N-(sec-butyl)-4-(pyridin-4-yl)benzamido)methyl)phenoxy)hexanoic acid; 6-(2-((N-(sec-butyl)-4-(1H-pyrazol-4-yl)benzamido)methyl)phenoxy)hexanoic acid; 6-(2-((N-(sec-butyl)-4-(furan-2-yl)benzamido)methyl)phenoxy)hexanoic acid; 6-(2-((N-(sec-butyl)-4-(furan-3-yl)benzamido)methyl)phenoxy)hexanoic acid; 6-(2-((N-(sec-butyl)-4-(thiophen-2-yl)benzamido)methyl)phenoxy)hexanoic acid; 6-(2-((N-(sec-butyl)-4-(thiophen-3-yl)benzamido)methyl)phenoxy)hexanoic acid; 6-(2-((4-bromo-N-(sec-butyl)benzamido)methyl)phenoxy)hexanoic acid; 6-(2-((N-(3-morpholinopropyl)-4-(pyridin-3-yl)benzamido)methyl)phenoxy)hexanoic acid; 6-(2-((N-(3-morpholinopropyl)-4-(pyridin-4-yl)benzamido)methyl)phenoxy)hexanoic acid; 6-(2-((N-(3-morpholinopropyl)[1,1′-biphenyl]-4-carboxamido)methyl)phenoxy)hexanoic acid; 6-(2-((N-(3-morpholinopropyl)-4-(1H-pyrazol-4-yl)benzamido)methyl)phenoxy)hexanoic acid; 6-(2-((4-(furan-3-yl)-N-(3-morpholinopropyl)benzamido)methyl)phenoxy)hexanoic acid; 6-(2-((N-(3-morpholinopropyl)-4-(1H-pyrazol-4-yl)benzamido)methyl)phenoxy)hexanoic acid; 6-(2-((4-(furan-2-yl)-N-(3-morpholinopropyl)benzamido)methyl)phenoxy)hexanoic acid; 6-(2-((N-(2-(pyridin-2-yl)ethyl)[1,1′-biphenyl]-4-carboxamido)methyl)phenoxy)hexanoic acid; 6-(2-((N-(3-morpholinopropyl)-4-(thiophen-3-yl)benzamido)methyl)phenoxy)hexanoic acid; 6-(2-((N-(3-morpholinopropyl)-4-(thiophen-2-yl)benzamido)methyl)phenoxy)hexanoic acid; 6-(2-((N-(2-(pyridin-2-yl)ethyl)-4-(pyridin-3-yl)benzamido)methyl)phenoxy)hexanoic acid; 6-(2-((N-(2-(pyridin-2-yl)ethyl)-4-(pyridin-4-yl)benzamido)methyl)phenoxy)hexanoic acid; 6-(2-((4-(1H-pyrazol-4-yl)-N-(2-(pyridin-2-yl)ethyl)benzamido)methyl)phenoxy)hexanoic acid; 6-(2-((4-(furan-3-yl)-N-(2-(pyridin-2-yl)ethyl)benzamido)methyl)phenoxy)hexanoic acid; 6-(2-((N-(2-(pyridin-2-yl)ethyl)-4-(thiophen-2-yl)benzamido)methyl)phenoxy)hexanoic acid; 6-(2-((4-(furan-2-yl)-N-(2-(pyridin-2-yl)ethyl)benzamido)methyl)phenoxy)hexanoic acid; 6-(2-((N-(2-(pyridin-2-yl)ethyl)-4-(thiophen-3-yl)benzamido)methyl)phenoxy)hexanoic acid; 6-(2-((N-isopropyl-4-(pyridin-4-yl)benzamido)methyl)phenoxy)hexanoic acid; 6-(2-((N-isopropyl-4-(1H-pyrazol-4-yl)benzamido)methyl)phenoxy)hexanoic acid; 6-(2-((4-(furan-2-yl)-N-isopropylbenzamido)methyl)phenoxy)hexanoic acid; 6-(2-((N-isopropyl-4-(pyridin-3-yl)benzamido)methyl)phenoxy)hexanoic acid; 6-(2-((N-isopropyl-4-(thiophen-3-yl)benzamido)methyl)phenoxy)hexanoic acid; 6-(2-((4-(furan-3-yl)-N-isopropylbenzamido)methyl)phenoxy)hexanoic acid; 6-(2-((N-cyclopentyl-4-(pyridin-3-yl)benzamido)methyl)phenoxy)hexanoic acid; 6-(2-((N-cyclopentyl-[1,1′-biphenyl]-4-carboxamido)methyl)phenoxy)hexanoic acid; 6-(2-((N-isopropyl-4-(thiophen-2-yl)benzamido)methyl)phenoxy)hexanoic acid; 6-(2-((N-cyclopentyl-4-(1H-pyrazol-4-yl)benzamido)methyl)phenoxy)hexanoic acid; 6-(2-((N-cyclopentyl-4-(pyridin-4-yl)benzamido)methyl)phenoxy)hexanoic acid; 6-(2-((N-cyclopentyl-4-(furan-3-yl)benzamido)methyl)phenoxy)hexanoic acid; 6-(2-((N-cyclopentyl-4-(thiophen-3-yl)benzamido)methyl)phenoxy)hexanoic acid; 6-(2-((N-cyclopentyl-4-(furan-2-yl)benzamido)methyl)phenoxy)hexanoic acid; 6-(2-((N-cyclopropyl-4-(naphthalen-2-yl)benzamido)methyl)phenoxy)hexanoic acid; 6-(2-((N-cyclopropyl-4-(naphthalen-1-yl)benzamido)methyl)phenoxy)hexanoic acid; 6-(2-((N-cyclopropyl-2′-methyl-[1,1′-biphenyl]-4-carboxamido)methyl)phenoxy)hexanoic acid; 6-(2-((N-cyclopropyl-3′-methyl-[1,1′-biphenyl]-4-carboxamido)methyl)phenoxy)hexanoic acid; 6-(2-((N-cyclopropyl-2′-methoxy-[1,1′-biphenyl]-4-carboxamido)methyl)phenoxy)hexanoic acid; 6-(2-((N-cyclopropyl-4′-methoxy-[1,1′-biphenyl]-4-carboxamido)methyl)phenoxy)hexanoic acid; 6-(2-((N-cyclopropyl-3′-methoxy-[1,1′-biphenyl]-4-carboxamido)methyl)phenoxy)hexanoic acid; 6-(2-((N-cyclopropyl-4′-methyl-[1,1′-biphenyl]-4-carboxamido)methyl)phenoxy)hexanoic acid; 6-(2-((N-cyclopropyl-2′-fluoro-[1,1′-biphenyl]-4-carboxamido)methyl)phenoxy)hexanoic acid; 6-(2-((N-cyclopropyl-3′-fluoro-[1,1′-biphenyl]-4-carboxamido)methyl)phenoxy)hexanoic acid; 6-(2-((N-cyclopropyl-4′-fluoro-[1,1′-biphenyl]-4-carboxamido)methyl)phenoxy)hexanoic acid; 6-(2-((N-cyclopropyl-4′-ethyl-[1,1′-biphenyl]-4-carboxamido)methyl)phenoxy)hexanoic acid; 6-(2-((N-cyclopropyl-2′,3′-dimethyl-[1,1′-biphenyl]-4-carboxamido)methyl)phenoxy)hexanoic acid; 6-(2-((N-cyclopropyl-2′-ethyl-[1,1′-biphenyl]-4-carboxamido)methyl)phenoxy)hexanoic acid; 6-(2-((N-cyclopropyl-2′,5′-dimethyl-[1,1′-biphenyl]-4-carboxamido)methyl)phenoxy)hexanoic acid; 6-(2-((N-cyclopropyl-2′,6′-dimethyl-[1,1′-biphenyl]-4-carboxamido)methyl)phenoxy)hexanoic acid; 6-(2-((N-cyclopropyl-3′,5′-dimethyl-[1,1′-biphenyl]-4-carboxamido)methyl)phenoxy)hexanoic acid; 6-(2-((N-cyclopropyl-3′-(trifluoromethyl)-[1,1′-biphenyl]-4-carboxamido)methyl)phenoxy)hexanoic acid; 6-(2-((N-cyclopropyl-2′-(trifluoromethyl)-[1,1′-biphenyl]-4-carboxamido)methyl)phenoxy)hexanoic acid; 6-(2-((N-cyclopropyl-4′-(trifluoromethyl)-[1,1′-biphenyl]-4-carboxamido)methyl)phenoxy)hexanoic acid; 6-(2-((N-cyclopropyl-[1,1′:2′,1″-terphenyl]-4-carboxamido)methyl)phenoxy)hexanoic acid; 6-(2-((N-cyclopropyl-4′-propyl-[1,1′-biphenyl]-4-carboxamido)methyl)phenoxy)hexanoic acid; 6-(2-((4′-butyl-N-cyclopropyl-[1,1′-biphenyl]-4-carboxamido)methyl)phenoxy)hexanoic acid; 6-(2-((N-sec-butyl-4-(furan-2-yl)benzamido)methyl)phenoxy)hexanoic acid; 6-(2-((N-isopropylbiphenyl-4-ylcarboxamido)methyl)phenoxy)hexanoic acid; 6-(2-(2-(4-(furan-2-yl)phenyl)thiazol-5-yl)phenoxy)hexanoic acid; 6-(2-(cyclopropyl(4-(furan-2-yl)benzyl)carbamoyl)phenoxy)hexanoic acid; 6-(2-((N-cyclopropyl-2-oxoindoline-5-carboxamido)methyl)phenoxy)hexanoic acid; 6-(2-((N-cyclopropyl-2-oxo-2,3-dihydrobenzofuran-5-carboxamido)methyl)phenoxy)hexanoic acid; 6-(2-((N-cyclopropylbenzo[c][1,2,5]oxadiazole-5-carboxamido)methyl)phenoxy)hexanoic acid; 6-(2-((N-cyclopropyl-5-(furan-2-yl)thiazole-2-carboxamido)methyl)phenoxy)hexanoic acid; 6-(2-((N-cyclopropyl-[2,3′-bifuran]-F-carboxamido)methyl)phenoxy)hexanoic acid; 6-((4-((N-cyclopropyl-4-(furan-2-yl)benzamido)methyl)thiophen-3-yl)oxy)hexanoic acid; N-(2-((5-(1H-tetrazol-5-yl)pentyl)oxy)benzyl)-N-cyclopropyl-4-(furan-2-yl)benzamide; 6-(2-((N-cyclopropyl-4-(furan-2-yl)benzamido)methyl)phenoxy)hex-3-ynoic acid; 6-(2-((N-cyclopropyl-4-(furan-2-yl)benzamido)methyl)phenoxy)hex-4-ynoic acid; (Z)-6-(2-((N-cyclopropyl-4-(furan-2-yl)benzamido)methyl)phenoxy)hex-3-enoic acid; (E)-6-(2-((N-cyclopropyl-4-(furan-2-yl)benzamido)methyl)phenoxy)hex-4-enoic acid; (E)-6-(2-((N-cyclopropyl-4-(furan-2-yl)benzamido)methyl)phenoxy)hex-2-enoic acid; (Z)-6-(2-((N-cyclopropyl-4-(furan-2-yl)benzamido)methyl)phenoxy)hex-4-enoic acid; (Z)-6-(2-((N-cyclopropyl-4-(furan-2-yl)benzamido)methyl)phenoxy)hex-2-enoic acid; (E)-6-(2-((N-cyclopropyl-4-(furan-2-yl)benzamido)methyl)phenoxy)hex-3-enoic acid; 6-(2-((N-isopropyl-[1,1′-biphenyl]-4-carboxamido)methyl)phenoxy)hexanoic acid; ethyl 6-(2-(1-(4-bromo-N-cyclopropylbenzamido)-2-(tert-butylamino)-2-oxoethyl)phenoxy)hexanoate; ethyl 6-(2-(2-(tert-butylamino)-1-(N-cyclopropyl-[1,1′-biphenyl]-4-carboxamido)-2-oxoethyl)phenoxy)hexanoate; ethyl 6-(2-(2-amino-1-(N-cyclopropyl-[1,1′-biphenyl]-4-carboxamido)-2-oxoethyl)phenoxy)hexanoate; 6-(2-(2-amino-1-(N-cyclopropyl-[1,1′-biphenyl]-4-carboxamido)-2-oxoethyl)phenoxy)hexanoic acid; 6-(2-(2-(tert-butylamino)-1-(N-cyclopropyl-[1,1′-biphenyl]-4-carboxamido)-2-oxoethyl)phenoxy)hexanoic acid; 6-(2-((N-cyclopropyl-[1,1′-biphenyl]-4-carboxamido)methyl)phenoxy)hexanoic acid; N-(2-amino-1 -(2-((6-(hydroxyamino)-6-oxohexyl)oxy)phenyl)-2-oxoethyl)-N-cyclopropyl-[1,1′-biphenyl]-4-carboxamide; 6-(2-((N-cyclopropyl-4-(pyridin-4-yl)benzamido)methyl)phenoxy)hexanoic acid; 6-(2-((N-cyclopropyl-4-(pyridin-3-yl)benzamido)methyl)phenoxy)hexanoic acid; 6-(2-((N-cyclopropyl-4-(1H-pyrazol-4-yl)benzamido)methyl)phenoxy)hexanoic acid; 6-(2-((N-cyclopropyl-4-(furan-2-yl)benzamido)methyl)phenoxy)hexanoic acid; 6-(2-((N-cyclopropyl-4-(furan-3-yl)benzamido)methyl)phenoxy)hexanoic acid; 6-(2-((N-cyclopropyl-4-(thiophen-2-yl)benzamido)methyl)phenoxy)hexanoic acid; 6-(2-((N-cyclopropyl-4-(thiophen-3-yl)benzamido)methyl)phenoxy)hexanoic acid; 6-(2-((N-benzyl-[1,1′-biphenyl]-4-carboxamido)methyl)phenoxy)hexanoic acid; 6-(2-((N-benzyl-4-(pyridin-3-yl)benzamido)methyl)phenoxy)hexanoic acid; 6-(2-((N-benzyl-4-(pyridin-4-yl)benzamido)methyl)phenoxy)hexanoic acid; 6-(2-((N-benzyl-4-(1H-pyrazol-4-yl)benzamido)methyl)phenoxy)hexanoic acid; 6-(2-((N-benzyl-4-(thiophen-2-yl)benzamido)methyl)phenoxy)hexanoic acid; 6-(2-((N-benzyl-4-(furan-3-yl)benzamido)methyl)phenoxy)hexanoic acid; 6-(2-((N-benzyl-4-(furan-2-yl)benzamido)methyl)phenoxy)hexanoic acid; 6-(2-((N-benzyl-4-(thiophen-3-yl)benzamido)methyl)phenoxy)hexanoic acid; N-benzyl-N-(2-((6-(hydroxyamino)-6-oxohexyhoxy)benzyl)-4-(pyridin-4-yl)benzamide; N-benzyl-N-(2-((6-(hydroxyamino)-6-oxohexyhoxy)benzyl)-[1,1′-biphenyl]-4-carboxamide; N-benzyl-N-(2-((6-(hydroxyamino)-6-oxohexyhoxy)benzyl)-4-(pyridin-3-yl)benzamide; N-benzyl-N-(2-((6-(hydroxyamino)-6-oxohexyl)oxy)benzyl)-4-(1H-pyrazol-4-yl)benzamide; N-benzyl-N-(2-((6-(hydroxyamino)-6-oxohexyhoxy)benzyl)-4-(thiophen-2-yl)benzamide; N-benzyl-4-(furan-2-yl)-N-(2-((6-(hydroxyamino)-6-oxohexyl)oxy)benzyl)benzamide; N-benzyl-4-(furan-3-yl)-N-(2-((6-(hydroxyamino)-6-oxohexyl)oxy)benzyl)benzamide; 6-(2-((N-(sec-butyl)-[1,1′-biphenyl]-4-carboxamido)methyl)phenoxy)hexanoic acid; N-benzyl-N-(2-((6-(hydroxyamino)-6-oxohexyhoxy)benzyl)-4-(thiophen-3-yl)benzamide; 6-(2-((N-(sec-butyl)-4-(pyridin-3-yl)benzamido)methyl)phenoxy)hexanoic acid; 6-(2-((N-(sec-butyl)-4-(pyridin-4-yl)benzamido)methyl)phenoxy)hexanoic acid; 6-(2-((N-(sec-butyl)-4-(1H-pyrazol-4-yl)benzamido)methyl)phenoxy)hexanoic acid; 6-(2-((N-(sec-butyl)-4-(furan-2-yl)benzamido)methyl)phenoxy)hexanoic acid; 6-(2-((N-(sec-butyl)-4-(furan-3-yl)benzamido)methyl)phenoxy)hexanoic acid; 6-(2-((N-(sec-butyl)-4-(thiophen-2-yl)benzamido)methyl)phenoxy)hexanoic acid; 6-(2-((N-(sec-butyl)-4-(thiophen-3-yl)benzamido)methyl)phenoxy)hexanoic acid; 6-(2-((4-bromo-N-(sec-butyl)benzamido)methyl)phenoxy)hexanoic acid; 6-(2-((N-(3-morpholinopropyl)-4-(pyridin-3-yl)benzamido)methyl)phenoxy)hexanoic acid; 6-(2-((N-(3-morpholinopropyl)-4-(pyridin-4-yl)benzamido)methyl)phenoxy)hexanoic acid; 6-(2-((N-(3-morpholinopropyl)-[1,1′-biphenyl]-4-carboxamido)methyl)phenoxy)hexanoic acid; 6-(2-((N-(3-morpholinopropyl)-4-(1H-pyrazol-4-yl)benzamido)methyl)phenoxy)hexanoic acid; 6-(2-((4-(furan-3-yl)-N-(3-morpholinopropyl)benzamido)methyl)phenoxy)hexanoic acid; 6-(2-((N-(3-morpholinopropyl)-4-(1H-pyrazol-4-yl)benzamido)methyl)phenoxy)hexanoic acid; 6-(2-((4-(furan-2-yl)-N-(3-morpholinopropyl)benzamido)methyl)phenoxy)hexanoic acid; 6-(2-((N-(2-(pyridin-2-yl)ethyl)-[1,1′-biphenyl]-4-carboxamido)methyl)phenoxy)hexanoic acid; 6-(2-((N-(3-morpholinopropyl)-4-(thiophen-3-yl)benzamido)methyl)phenoxy)hexanoic acid; 6-(2-((N-(3-morpholinopropyl)-4-(thiophen-2-yl)benzamido)methyl)phenoxy)hexanoic acid; 6-(2-((N-(2-(pyridin-2-yl)ethyl)-4-(pyridin-3-yl)benzamido)methyl)phenoxy)hexanoic acid; 6-(2-((N-(2-(pyridin-2-yl)ethyl)-4-(pyridin-4-yl)benzamido)methyl)phenoxy)hexanoic acid; 6-(2-((4-(1H-pyrazol-4-yl)-N-(2-(pyridin-2-yl)ethyl)benzamido)methyl)phenoxy)hexanoic acid; 6-(2-((4-(furan-3-yl)-N-(2-(pyridin-2-yl)ethyl)benzamido)methyl)phenoxy)hexanoic acid; 6-(2-((N-(2-(pyridin-2-yl)ethyl)-4-(thiophen-2-yl)benzamido)methyl)phenoxy)hexanoic acid; 6-(2-((4-(furan-2-yl)-N-(2-(pyridin-2-yl)ethyl)benzamido)methyl)phenoxy)hexanoic acid; 6-(2-((N-(2-(pyridin-2-yl)ethyl)-4-(thiophen-3-yl)benzamido)methyl)phenoxy)hexanoic acid; 6-(2-((N-isopropyl-[1,1′-biphenyl]-4-carboxamido)methyl)phenoxy)hexanoic acid; 6-(2-((N-isopropyl-4-(pyridin-4-yl)benzamido)methyl)phenoxy)hexanoic acid; 6-(2-((N-isopropyl-4-(1H-pyrazol-4-yl)benzamido)methyl)phenoxy)hexanoic acid; 6-(2-((4-(furan-2-yl)-N-isopropylbenzamido)methyl)phenoxy)hexanoic acid; 6-(2-((N-isopropyl-4-(pyridin-3-yl)benzamido)methyl)phenoxy)hexanoic acid; 6-(2-((N-isopropyl-4-(thiophen-3-yl)benzamido)methyl)phenoxy)hexanoic acid; 6-(2-((4-(furan-3-yl)-N-isopropylbenzamido)methyl)phenoxy)hexanoic acid; 6-(2-((N-cyclopentyl-4-(pyridin-3-yl)benzamido)methyl)phenoxy)hexanoic acid; 6-(2-((N-cyclopentyl-[1,1′-biphenyl]-4-carboxamido)methyl)phenoxy)hexanoic acid; 6-(2-((N-isopropyl-4-(thiophen-2-yl)benzamido)methyl)phenoxy)hexanoic acid; 6-(2-((N-cyclopentyl-4-(1H-pyrazol-4-yl)benzamido)methyl)phenoxy)hexanoic acid; 6-(2-((N-cyclopentyl-4-(pyridin-4-yl)benzamido)methyl)phenoxy)hexanoic acid; 6-(2-((N-cyclopentyl-4-(furan-3-yl)benzamido)methyl)phenoxy)hexanoic acid; 6-(2-((N-cyclopentyl-4-(thiophen-3-yl)benzamido)methyl)phenoxy)hexanoic acid; 6-(2-((N-cyclopentyl-4-(furan-2-yl)benzamido)methyl)phenoxy)hexanoic acid; 6-(2-((N-cyclopropyl-4-(naphthalen-2-yl)benzamido)methyl)phenoxy)hexanoic acid; 6-(2-((N-cyclopropyl-4-(naphthalen-1-yl)benzamido)methyl)phenoxy)hexanoic acid; 6-(2-((N-cyclopropyl-2′-methyl-[1,1′-biphenyl]-4-carboxamido)methyl)phenoxy)hexanoic acid; 6-(2-((N-cyclopropyl-3′-methyl-[1,1′-biphenyl]-4-carboxamido)methyl)phenoxy)hexanoic acid; 6-(2-((N-cyclopropyl-2′-methoxy-[1,1′-biphenyl]-4-carboxamido)methyl)phenoxy)hexanoic acid; 6-(2-((N-cyclopropyl-4′-methoxy-[1,1′-biphenyl]-4-carboxamido)methyl)phenoxy)hexanoic acid; 6-(2-((N-cyclopropyl-3′-methoxy-[1,1′-biphenyl]-4-carboxamido)methyl)phenoxy)hexanoic acid; 6-(2-((N-cyclopropyl-4′-methyl-[1,1′-biphenyl]-4-carboxamido)methyl)phenoxy)hexanoic acid; 6-(2-((N-cyclopropyl-2′-fluoro-[1,1′-biphenyl]-4-carboxamido)methyl)phenoxy)hexanoic acid; 6-(2-((N-cyclopropyl-3′-fluoro-[1,1′-biphenyl]-4-carboxamido)methyl)phenoxy)hexanoic acid; 6-(2-((N-cyclopropyl-4′-fluoro-[1,1′-biphenyl]-4-carboxamido)methyl)phenoxy)hexanoic acid; 6-(2-((N-cyclopropyl-4′-ethyl-[1,1′-biphenyl]-4-carboxamido)methyl)phenoxy)hexanoic acid; 6-(2-((N-cyclopropyl-2′,3′-dimethyl-[1,1′-biphenyl]-4-carboxamido)methyl)phenoxy)hexanoic acid; 6-(2-((N-cyclopropyl-2′-ethyl-[1,1′-biphenyl]-4-carboxamido)methyl)phenoxy)hexanoic acid; 6-(2-((N-cyclopropyl-2′,5′-dimethyl-[1,1′-biphenyl]-4-carboxamido)methyl)phenoxy)hexanoic acid; 6-(2-((N-cyclopropyl-2′,6′-dimethyl-[1,1′-biphenyl]-4-carboxamido)methyl)phenoxy)hexanoic acid; 6-(2-((N-cyclopropyl-3′,5′-dimethyl-[1,1′-biphenyl]-4-carboxamido)methyl)phenoxy)hexanoic acid; 6-(2-((N-cyclopropyl-3′-(trifluoromethyl)-[1,1′-biphenyl]-4-carboxamido)methyl)phenoxy)hexanoic acid; 6-(2-((N-cyclopropyl-2′-(trifluoromethyl)-[1,1′-biphenyl]-4-carboxamido)methyl)phenoxy)hexanoic acid; 6-(2-((N-cyclopropyl-4′-(trifluoromethyl)-[1,1′-biphenyl]-4-carboxamido)methyl)phenoxy)hexanoic acid; 6-(2-((N-cyclopropyl-[1,1′:2′,1″-terphenyl]-4-carboxamido)methyl)phenoxy)hexanoic acid; 6-(2-((N-cyclopropyl-4′-propyl-[1,1′-biphenyl]-4-carboxamido)methyl)phenoxy)hexanoic acid; 6-(2-((4′-butyl-N-cyclopropyl-[1,1′-biphenyl]-4-carboxamido)methyl)phenoxy)hexanoic acid; 6-(2-((N-sec-butyl-4-(furan-2-yl)benzamido)methyl)phenoxy)hexanoic acid; 6-(2-((N-isopropylbiphenyl-4-ylcarboxamido)methyl)phenoxy)hexanoic acid; 6-(2-(2-(4-(furan-2-yl)phenyl)thiazol-5-yl)phenoxy)hexanoic acid; 6-(2-(cyclopropyl(4-(furan-2-yl)benzyl)carbamoyl)phenoxy)hexanoic acid; 6-(2-((N-cyclopropyl-2-oxoindoline-5-carboxamido)methyl)phenoxy)hexanoic acid; 6-(2-((N-cyclopropylbenzo[c][1,2,5]oxadiazole-5-carboxamido)methyl)phenoxy)hexanoic acid; 6-((4-((N-cyclopropyl-4-(furan-2-yl)benzamido)methyl)thiophen-3-yl)oxy)hexanoic acid; N-(2-((5-(1H-tetrazol-5-yl)pentyl)oxy)benzyl)-N-cyclopropyl-4-(furan-2-yl)benzamide; 6-(2-((N-cyclopropyl-4-(furan-2-yl)benzamido)methyl)phenoxy)hex-3-ynoic acid; 6-(2-((N-cyclopropyl-4-(furan-2-yl)benzamido)methyl)phenoxy)hex-4-ynoic acid; (Z)-6-(2-((N-cyclopropyl-4-(furan-2-yl)benzamido)methyl)phenoxy)hex-3-enoic acid; (E)-6-(2-((N-cyclopropyl-4-(furan-2-yl)benzamido)methyl)phenoxy)hex-4-enoic acid; (E)-6-(2-((N-cyclopropyl-4-(furan-2-yl)benzamido)methyl)phenoxy)hex-2-enoic acid; (Z)-6-(2-((N-cyclopropyl-4-(furan-2-yl)benzamido)methyl)phenoxy)hex-4-enoic acid; (Z)-6-(2-((N-cyclopropyl-4-(furan-2-yl)benzamido)methyl)phenoxy)hex-2-enoic acid; (E)-6-(2-((N-cyclopropyl-4-(furan-2-yl)benzamido)methyl)phenoxy)hex-3-enoic acid; N-cyclopropyl-N-(2-((5-(2,4-dioxothiazolidin-5-yl)pentypoxy)benzyl)-4-(furan-2-yl)benzamide; N-cyclopropyl-N-(2-((5-(2,4-dioxooxazolidin-5-yl)pentypoxy)benzyl)-4-(furan-2-yl)benzamide; N-cyclopropyl-4-(furan-2-yl)-N-(2-((5-(3-hydroxy-1-methyl-1H-pyrazol-5-yl)pentyl)oxy)benzyl)benzamide; N-cyclopropyl-4-(furan-2-yl)-N-(2-((5-(3-hydroxyisothiazol-5-yl)pentyl)oxy)benzyl)benzamide; N-cyclopropyl-4-(furan-2-yl)-N-(2-((5-(3-hydroxyisoxazol-5-yl)pentyl)oxy)benzyl)benzamide; N-cyclopropyl-N-(2-((5-(2,5-dioxo-2,5-dihydro-1H-imidazol-4-yl)pentypoxy)benzyl)-4-(furan-2-yl)benzamide; N-cyclopropyl-N-(2-((5-(2,5-dioxo-2,5-dihydro-1H-pyrrol-3-yl)pentypoxy)benzyl)-4-(furan-2-y1)benzamide; N-cyclopropyl-4-(furan-2-yl)-N-(2-((5-(6-hydroxy-4-oxo-4H-1,3-dioxin-2-yl)pentyl)oxy)benzyl)benzamide; 6-(2-((4-cyclopropoxy-N-isopropylbenzamido)methyl)phenoxy)hexanoic acid; 6-(2-((N-isopropyl-4-methylbenzamido)methyl)phenoxy)hexanoic acid; 6-(2-((4-(cyclopentylethynyl)-N-isopropylbenzamido)methyl)phenoxy)hexanoic acid; 6-(2-((N-isopropyl-4-((1-methylazetidin-3-yl)ethynyl)benzamido)methyl)phenoxy)hexanoic acid; 6-(2-((4-chloro-N-isopropylbenzamido)methyl)phenoxy)hexanoic acid; 6-(2-((N-isopropyl-4-methoxybenzamido)methyl)phenoxy)hexanoic acid; 6-(2-((4-(dimethylamino)-N-isopropylbenzamido)methyl)phenoxy)hexanoic acid; 6-(2-((N-isopropyl-4-(trifluoromethoxy)benzamido)methyl)phenoxy)hexanoic acid; 6-(2-((4-acetyl-N-isopropylbenzamido)methyl)phenoxy)hexanoic acid; 6-(2-((N-isopropyl-4-(methylsulfonyl)benzamido)methyl)phenoxy)hexanoic acid; 6-(2-((3′-(furan-3-yl)-N-isopropyl-[1,1′-biphenyl]-4-carboxamido)methyl)phenoxy)hexanoic acid; 6-(2-((4-fluoro-N-isopropylbenzamido)methyl)phenoxy)hexanoic acid; 6-(2-((N-isopropyl-4-(4-methoxytetrahydro-2H-pyran-4-yl)benzamido)methyl)phenoxy)hexanoic acid; 6-(2-((N-isopropyl-4-(3,3,3-trifluoroprop-1-yn-1-yl)benzamido)methyl)phenoxy)hexanoic acid; 6-(2-((N-isopropyl-4-(oxetan-3-ylethynyl)benzamido)methyl)phenoxy)hexanoic acid; 6-(2-((4-(cyclobutylethynyl)-N-isopropylbenzamido)methyl)phenoxy)hexanoic acid; 6-(2-((N-isopropyl-4-(1-(trifluoromethyl)cyclopropyl)benzamido)methyl)phenoxy)hexanoic acid; 6-(2-((N-isopropyl-4-(1-methoxycyclopropyl)benzamido)methyl)phenoxy)hexanoic acid; 6-(2-((N-isopropyl-4-(tetrahydro-2H-pyran-2-yl)benzamido)methyl)phenoxy)hexanoic acid; 6-(2-((N-isopropyl-4-(4-methylbicyclo[2.2.2]octan-1-yl)benzamido)methyl)phenoxy)hexanoic acid; 6-(2-((N-isopropyl-4-(6-oxo-1,6-dihydropyridin-2-yl)benzamido)methyl)phenoxy)hexanoic acid; 6-(2-((N-isopropyl-4-(oxetan-2-ylethynyl)benzamido)methyl)phenoxy)hexanoic acid; 6-(2-((N-isopropyl-4-(4-(trifluoromethyl)bicyclo[2.2.2]octan-1-yl)benzamido)methyl)phenoxy)hexanoic acid; 6-(2-((N-isopropyl-4-(4-phenylbicyclo[2.2.2]octan-1-yl)benzamido)methyl)phenoxy)hexanoic acid; 6-(2-((4-cyano-N-isopropylbenzamido)methyl)phenoxy)hexanoic acid; 6-(2-((N-isopropyl-4-(oxetan-2-yl)benzamido)methyl)phenoxy)hexanoic acid; 6-(2-((N-isopropyl-4-(pyrrolidin-1-yl)benzamido)methyl)phenoxy)hexanoic acid; 6-(2-((5-(furan-2-yl)-N-isopropylpicolinamido)methyl)phenoxy)hexanoic acid; 6-(2-((2-(furan-2-yl)-N-isopropylthiazole-5-carboxamido)methyl)phenoxy)hexanoic acid; 6-(2-((4-(furan-2-yl)-N-isopropyl-2,5-dioxopiperazine-1-carboxamido)methyl)phenoxy)hexanoic acid; 6-(2-(((4-(furan-2-yl)-N-isopropylphenyl)sulfonamido)methyl)phenoxy)hexanoic acid; 6-(2-(2-((4-(furan-2-yl)phenyl)(isopropyl)amino)-2-oxoethyl)phenoxy)hexanoic acid; 6-(2-((6-(furan-2-yl)-N-isopropylnicotinamido)methyl)phenoxy)hexanoic acid; 6-(2-((4-(furan-2-yl)benzyl)(isopropyl)carbamoyl)phenoxy)hexanoic acid; 6-(2-((2-(furan-2-yl)-N-isopropyloxazole-5-carboxamido)methyl)phenoxy)hexanoic acid; 6-(2-(3-((4-(furan-2-yl)phenyl)(isopropyl)amino)-3-oxopropyl)phenoxy)hexanoic acid; 6-(2-((2-fluoro-4-(furan-2-yl)-N-isopropylbenzamido)methyl)phenoxy)hexanoic acid; 6-(2-((3-fluoro-4-(furan-2-yl)-N-isopropylbenzamido)methyl)phenoxy)hexanoic acid; 6-(2-((1-(furan-2-yl)-N-isopropylpiperidine-4-carboxamido)methyl)phenoxy)hexanoic acid; 6-(2-((5-(furan-2-yl)-N-isopropylisoxazole-3-carboxamido)methyl)phenoxy)hexanoic acid; 6-(2-((4-(furan-2-yl)-N-isopropylcyclohexane-1-carboxamido)methyl)phenoxy)hexanoic acid; 6-(2-(((6-(furan-2-yl)-1H-indazol-3-yl)(isopropyl)amino)methyl)phenoxy)hexanoic acid; 6-(2-((5-(furan-2-yl)-N-isopropylthiazole-2-carboxamido)methyl)phenoxy)hexanoic acid; 6-(2-((N-isopropyl-2-methylbenzofuran-6-carboxamido)methyl)phenoxy)hexanoic acid; 6-(2-((N-isopropyl-2-methylbenzofuran-5-carboxamido)methyl)phenoxy)hexanoic acid; 6-(2-((7-(furan-2-yl)-4-oxoquinazolin-3(4H)-yl)methyl)phenoxy)hexanoic acid; 6-(2-((1-(furan-2-yl)-N-isopropyl-2-oxopiperidine-4-carboxamido)methyl)phenoxy)hexanoic acid; 6-(2-((5-(furan-2-yl)-N-isopropyl-1H-pyrazole-3-carboxamido)methyl)phenoxy)hexanoic acid; 6-(2-((5-(furan-2-yl)-N-isopropyl-1-methyl-1H-pyrazole-3-carboxamido)methyl)phenoxy)hexanoic acid; 6-(2-((5-(furan-2-yl)-N-isopropyl-3,6-dioxopiperazine-2-carboxamido)methyl)phenoxy)hexanoic acid; 6-(2-((4-(furan-2-yl)-N-isopropylpiperidine-1-carboxamido)methyl)phenoxy)hexanoic acid; 6-(2-((4-(furan-2-yl)-N-methylbenzamido)methyl)phenoxy)hexanoic acid; 6-(2-((4-(furan-2-yl)-N-(2,2,2-trifluoroethyl)benzamido)methyl)phenoxy)hexanoic acid; 6-(2-((4-(furan-2-yl)-N-(2-methoxyethyl)benzamido)methyl)phenoxy)hexanoic acid; 6-(2-((4-(furan-2-yl)benzamido)methyl)phenoxy)hexanoic acid; 6-(2-((4-(furan-2-yl)-N-(oxetan-3-yl)benzamido)methyl)phenoxy)hexanoic acid; 6-(2-((2-(4-(furan-2-yl)phenyl)-5-methyl-1H-imidazol-1-yl)methyl)phenoxy)hexanoic acid; 6-(2-((N-(2-cyanopropan-2-yl)-4-(furan-2-yl)benzamido)methyl)phenoxy)hexanoic acid; 6-(2-((2-(4-(furan-2-yl)phenyl)-4-methyl-1H-imidazol-1-yl)methyl)phenoxy)hexanoic acid; 6-(2-(2-(4-(furan-2-yl)phenyl)-1-methyl-1H-imidazol-5-yl)phenoxy)hexanoic acid; 6-(2-((6-(furan-2-yl)-3-methyl-1-oxo-3,4-dihydroisoquinolin-2(1H)-yl)methyl)phenoxy)hexanoic acid; 6-(2-((4-(furan-2-yl)-N-hydroxybenzamido)methyl)phenoxy)hexanoic acid; 6-(2-((3-(4-(furan-2-yl)phenyl)-5-methylisoxazol-4-yl)methyl)phenoxy)hexanoic acid; 6-(2-((4-(furan-2-yl)-N-methoxybenzamido)methyl)phenoxy)hexanoic acid; 6-(2-((N-(cyclopropylmethyl)-4-(furan-2-yl)benzamido)methyl)phenoxy)hexanoic acid; 6-(2-((N-(1-cyclopropylethyl)-4-(furan-2-yl)benzamido)methyl)phenoxy)hexanoic acid; 6-(2-(1-(4-(furan-2-yl)benzoyl)pyrrolidin-2-yl)phenoxy)hexanoic acid; 6-(2-(1-(4-(furan-2-yl)benzoyl)azetidin-2-yl)phenoxy)hexanoic acid; 6-(2-(1-(4-(furan-2-yl)benzoyl)piperidin-2-yl)phenoxy)hexanoic acid; 6-(4-bromo-2-((4-(furan-2-yl)-N-isopropylbenzamido)methyl)phenoxy)hexanoic acid; 6-(2-((4-(furan-2-yl)-N-isopropylbenzamido)methyl)-4-methylphenoxy)hexanoic acid; 6-(4-fluoro-2-((4-(furan-2-yl)-N-isopropylbenzamido)methyl)phenoxy)hexanoic acid; 6-(4-cyano-2-((4-(furan-2-yl)-N-isopropylbenzamido)methyl)phenoxy)hexanoic acid; 6-(2-((4-(furan-2-yl)-N-isopropylbenzamido)methyl)-4-methoxyphenoxy)hexanoic acid; 6-((3-((4-(furan-2-yl)-N-isopropylbenzamido)methyl)pyridin-2-yl)oxy)hexanoic acid; 6-((2-((4-(furan-2-yl)-N-isopropylbenzamido)methyl)pyridin-3-yl)oxy)hexanoic acid; 6-((3-((4-(furan-2-yl)-N-isopropylbenzamido)methyl)pyridin-4-yl)oxy)hexanoic acid; 6-((4-((4-(furan-2-yl)-N-isopropylbenzamido)methyl)-1-methyl-1H-pyrazol-3-yl)oxy)hexanoic acid; 6-((4-((4-(furan-2-yl)-N-isopropylbenzamido)methyl)pyridin-3-yl)oxy)hexanoic acid; 6-((2-(4-(furan-2-yl)benzoyl)-1,2,3,4-tetrahydroisoquinolin-8-yl)oxy)hexanoic acid; 6-((4-((4-(furan-2-yl)-N-isopropylbenzamido)methyl)isothiazol-3-yl)oxy)hexanoic acid; 6-((2-((4-(furan-2-yl)-N-isopropylbenzamido)methyl)cyclopentyl)oxy)hexanoic acid; 6-(4-cyclopropyl-2-((4-(furan-2-yl)-N-isopropylbenzamido)methyl)phenoxy)hexanoic acid; 6-((2-((4-(furan-2-yl)-N-isopropylbenzamido)methyl)cyclohexyl)oxy)hexanoic acid; 6-(4-(azetidin-1-yl)-2-((4-(furan-2-yl)-N-isopropylbenzamido)methyl)phenoxy)hexanoic acid; 6-(2-((4-(furan-2-yl)-N-isopropylbenzamido)methyl)-4-(trifluoromethyl)phenoxy)hexanoic acid; N-(2-(4-(2H-tetrazol-5-yl)butoxy)benzyl)-4-(furan-2-yl)-N-isopropylbenzamide; 7-(2-((4-(furan-2-yl)-N-isopropylbenzamido)methyl)phenoxy)heptanoic acid; 2-(3-(2-((4-(furan-2-yl)-N-isopropylbenzamido)methyl)phenoxy)propoxy)acetic acid; 5-(2-(2-((4-(furan-2-yl)-N-isopropylbenzamido)methyl)phenoxy)ethyl)isoxazole-3-carboxylic acid; 2-(5-((2-((4-(furan-2-yl)-N-isopropylbenzamido)methyl)phenoxy)methyl)isoxazol-3-yl)acetic acid; 2-(4-(2-((4-(furan-2-yl)-N-isopropylbenzamido)methyl)phenoxy)cyclohexyl)acetic acid; 5-(2-((4-(furan-2-yl)-N-isopropylbenzamido)methyl)phenoxy)pentanoic acid; 3-(2-(2-((4-(furan-2-yl)-N-isopropylbenzamido)methyl)phenoxy)ethoxy)propanoic acid; 3-(2-(2-((4-(furan-2-yl)-N-isopropylbenzamido)methyl)phenoxy)acetamido)propanoic acid; 3-(4-((2-((4-(furan-2-yl)-N-isopropylbenzamido)methyl)phenoxy)methyl)thiazol-2-yl)propanoic acid; 3-(2-(2-((4-(furan-2-yl)-N-isopropylbenzamido)methyl)phenoxy)ethyl)cyclobutane-1-carboxylic acid; 3-((2-(2-((4-(furan-2-yl)-N-isopropylbenzamido)methyl)phenoxy)ethyl)amino)-3-oxopropanoic acid; 3-(3-(2-((4-(furan-2-yl)-N-isopropylbenzamido)methyl)phenoxy)azetidin-1-yl)propanoic acid; 6-(2-((4-(furan-2-yl)-N-isopropylbenzamido)methyl)phenoxy)heptanoic acid; 2-(3-((2-((4-(furan-2-yl)-N-isopropylbenzamido)methyl)phenoxy)methyl)azetidin-1-yl)acetic acid; 2-(4-((2-((4-(furan-2-yl)-N-isopropylbenzamido)methyl)phenoxy)methyl)thiazol-2-yl)acetic acid; 2-((3-(2-((4-(furan-2-yl)-N-isopropylbenzamido)methyl)phenoxy)propyl)thio)acetic acid; 2-((3-(2-((4-(furan-2-yl)-N-isopropylbenzamido)methyl)phenoxy)cyclopentyl)oxy)acetic acid; 1-(2-(2-((4-(furan-2-yl)-N-isopropylbenzamido)methyl)phenoxy)acetyl)pyrrolidine-3-carboxylic acid; 1-(2-(2-((4-(furan-2-yl)-N-isopropylbenzamido)methyl)phenoxy)ethyl)pyrrolidine-3-carboxylic acid; (E)-6-(2-((4-(furan-2-yl)-N-isopropylbenzamido)methyl)phenoxy)-4-methylhex-4-enoic acid; (S)-4-((2-((4-(furan-2-yl)-N-isopropylbenzamido)methyl)phenyl)sulfonyl)-2,3-dihydro-1H-indene-2-carboxylic acid; 4-(furan-2-yl)-N-(2-(4-(5-hydroxy-1,3,4-oxadiazol-2-yl)butoxy)benzyl)-N-isopropylbenzamide; 1-(2-(2-((4-(furan-2-yl)-N-isopropylbenzamido)methyl)phenoxy)ethyl)azetidine-3-carboxylic acid; 2-(4-((2-((4-(furan-2-yl)-N-isopropylbenzamido)methyl)phenoxy)methyl)-1H-1,2,3-triazol-1-yl)acetic acid; 3-(3-(2-((4-(furan-2-yl)-N-isopropylbenzamido)methyl)phenoxy)piperidin-1-yl)propanoic acid; 6-(2-((4-(cyclopropylethynyl)-N-isopropylbenzamido)methyl)phenoxy)hexanoic acid; 6-(2-(1-(4-(furan-2-yl)benzyl)-5-methyl-1H-imidazol-2-yl)phenoxy)hexanoic acid; 6-(2-((4-(4-(furan-2-yl)phenyl)-5-methylisoxazol-3-yl)methyl)phenoxy)hexanoic acid; 6-(2-((N-benzyl-4-(furan-2-yl)benzamido)methyl)phenoxy)hexanoic acid; 6-(2-((4-(cyclopropylethynyl)-N-methylbenzamido)methyl)phenoxy)hexanoic acid; 6-(2-((N-methyl-4-(3,3,3-trifluoroprop-1-yn-1-yl)benzamido)methyl)phenoxy)hexanoic acid; 6-(2-((4-cyclopropoxy-N-methylbenzamido)methyl)phenoxy)hexanoic acid; 6-(2-((5-(furan-2-yl)-N-methylpicolinamido)methyl)phenoxy)hexanoic acid; 6-(2-((5-(furan-2-yl)-N-methylthiazole-2-carboxamido)methyl)phenoxy)hexanoic acid; 3-(2-(2-((4-(furan-2-yl)-N-methylbenzamido)methyl)phenoxy)ethoxy)propanoic acid; N-(2-(4-(2H-tetrazol-5-yl)butoxy)benzyl)-4-(furan-2-yl)-N-methylbenzamide; 6-(2-((4-(furan-2-yl)-N-methylbenzamido)methyl)phenoxy)heptanoic acid; 6-((3-((4-(furan-2-yl)-N-methylbenzamido)methyl)pyridin-2-yl)oxy)hexanoic acid; 6-((3-((6-(furan-2-yl)-N-methylnicotinamido)methyl)pyridin-2-yl)oxy)hexanoic acid; 6-(4-fluoro-2-((4-(furan-2-yl)-N-methylbenzamido)methyl)phenoxy)hexanoic acid; 6-(2-((4-(furan-2-yl)-N-methylbenzamido)methyl)-4-methoxyphenoxy)hexanoic acid; 6-(2-((4-(furan-2-yl)-N-methylbenzamido)methyl)-4-methylphenoxy)hexanoic acid; 6-(2-((6-(cyclopropylethynyl)-N-isopropylnicotinamido)methyl)phenoxy)hexanoic acid; 6-(2-((6-(cyclopropylethynyl)-N-methylnicotinamido)methyl)phenoxy)hexanoic acid; 6-(2-((N-(but-2-yn-1-yl)-6-(furan-2-yl)nicotinamido)methyl)phenoxy)hexanoic acid; 6-(2-((6-fluoro-N-isopropylnicotinamido)methyl)phenoxy)hexanoic acid; 6-(2-((6-fluoro-N-(furan-2-ylmethyl)nicotinamido)methyl)phenoxy)hexanoic acid; 6-(2-((N-benzyl-6-fluoronicotinamido)methyl)phenoxy)hexanoic acid; 6-(2-(2-(4-(furan-2-yl)phenyl)pyrrolidine-1-carbonyl)phenoxy)hexanoic acid; 6-(2-((N-isopropyl-[1,1′-biphenyl]-4-carboxamido)methyl)phenoxy)hexanoic acid; 6-(2-((N-(sec-butyl)-4-(furan-2-yl)benzamido)methyl)phenoxy)hexanoic acid; 6-(2-((N-isopropyl-4-(thiophen-2-yl)benzamido)methyl)phenoxy)hexanoic acid; sodium 6-(2-((N-cyclopropyl-4-(furan-2-yl)benzamido)methyl)phenoxy)hexanoate; 2-(2-methyl-4-(((4-methyl-2-(4-(trifluoromethyl)phenyl)thiazol-5-yl)methyl)thio)phenoxy)acetic acid; (E)-6-(2-((4-(furan-2-yl)-N′-hydroxy-N-isopropylbenzimidamido)methyl)phenoxy)hexanoic acid; 6-(2-((4-((fluoromethyl)thio)-N-isopropylbenzamido)methyl)phenoxy)hexanoic acid; 6-(2-((4-((difluoromethyl)thio)-N-isopropylbenzamido)methyl)phenoxy)hexanoic acid; 6-(2-((4-fluoro-5-(furan-2-yl)-N-isopropyl-1H-pyrazole-3-carboxamido)methyl)phenoxy)hexanoic acid; 6-(2-((5-(cyclopropylethynyl)-N-isopropyl-1H-pyrazole-3-carboxamido)methyl)phenoxy)hexanoic acid; (R)-6-(2-((5-(furan-2-yl)-N-isopropyl-1H-pyrazole-3-carboxamido)methyl)phenoxy)heptanoic acid; 6-(2-((3-(4-(furan-2-yl)phenyl)-5-methyl-4H-1,2,4-triazol-4-yl)methyl)phenoxy)hexanoic acid; 6-(2-((5-(4-(furan-2-yl)phenyl)-3-methyl-1H-1,2,4-triazol-1-yl)methyl)phenoxy)hexanoic acid; 6-(2-((2-(5-(furan-2-yl)pyridin-2-yl)-4-methyl-1H-imidazol-1-yl)methyl)phenoxy)hexanoic acid; 6-(2-((2-(5-(furan-2-yl)pyridin-2-yl)-5-methyl-1H-imidazol-1-yl)methyl)phenoxy)hexanoic acid; 6-(2-((2-(4-(furan-2-yl)phenyl)-5-methyl-1H-imidazol-1-yl)methyl)phenoxy)hexanoic acid; 6-(2-((2-(4-(furan-2-yl)phenyl)-4-methyl-1H-imidazol-1-yl)methyl)phenoxy)hexanoic acid; 6-(2-(5-(4-(furan-2-yl)phenyl)-1-methyl-1H-imidazol-2-yl)phenoxy)hexanoic acid; 6-(2-((4-(furan-2-yl)benzyl)(isopropyl)carbamoyl)phenoxy)hexanoic acid; 6-(2-(2-((4-(furan-2-yl)phenyl)(isopropyl)amino)-2-oxoethyl)phenoxy)hexanoic acid; 6-(2-(2-(4-(furan-2-yl)phenyl)piperidine-1-carbonyl)phenoxy)hexanoic acid; 6-(2-(2-(4-(furan-2-yl)phenyl)azetidine-1-carbonyl)phenoxy)hexanoic acid; 6-(2-((3-(4-(furan-2-yl)benzoyl)isoxazol-4-yl)methyl)phenoxy)hexanoic acid; 6-((4-((4-(furan-2-yl)-N-isopropylbenzamido)methyl)pyrrolidin-3-yl)oxy)hexanoic acid; 6-((4-((4-(furan-2-yl)-N-isopropylbenzamido)methyl)morpholin-3-yl)oxy)hexanoic acid; and 6-(2-(1-(4-(furan-2-yl)benzyl)-4-methyl-1H-imidazol-2-yl)phenoxy)hexanoic acid (see, U.S. Patent Application Publication No. 20160023991).

Without wishing to be bound by the theory, it is believed that while the PPARδ agonist increases the expression of the genes coding for mitochondrial β-oxidation pathway mutant proteins that are unstable but partially active, trimetazidine or its derivatives, act to indirectly stabilize the PPARδ agonist-enhanced produced mutant protein(s). The combination treatment is thus superior to treating patients with either drug individually.

As shown below, PPARδ agonists act synergistically with TMZ compounds in increasing activity of β-oxidation pathway enzymes, such as VLCAD and MCAD. Therefore provided is a method of treating a patient having a disorder caused by a mutation, e.g. a missense mutation, in an enzyme of the β-oxidation pathway and/or fatty acid mitochondrial transport, in a patient comprising inhibiting activity of an enzyme of the β-oxidation pathway downstream in the β-oxidation pathway to the enzyme having the missense mutation, thereby increasing activity of the an enzyme of the β-oxidation pathway and/or fatty acid mitochondrial transport having the missense mutation and administering to the patient a PPARδ agonist when the enzyme activity is inhibited, e.g., with TMZ. In aspects, a combination dosage form is provided comprising an effective amount of a PPARδ agonist to increase β-oxidation activity in a patient, and trimetazidine, a derivative thereof, an analog thereof, an isostere thereof, a pharmaceutically-acceptable salt thereof, or a pharmaceutically-acceptable ester thereof.

### EXAMPLE

In vitro experimental results presented herein provide evidence for the potential efficacy of TMZ in treating patients with fatty acid β-oxidation disorders. VLCAD, MCAD, TFP deficiencies, caused by missense mutations in the ACADVL, ACADM, HADHA and/or HADHB genes, coding for the two ACAD enzymes and the TFP, respectively. The evidence presented includes: (1) Significant increase in VLCAD and MCAD enzyme activity in patient's cell extracts with all being proportional with the increasing amounts of TMZ in the cell culture medium. (2) Increase in in situ VLCAD, MCAD, and TFP protein components signal presence proportional to the increase in the presence TMZ in culture. (3) Key acylcarnitine metabolites measured in the culture media of VLCAD deficient cells have shown profiles consistent with improvements that were best at 0.25 μM TMZ in one patient cell line tested. The preliminary results for TMZ effect warrant filing a patent while expanding the in vitro and in vivo investigation with other TMZ derivatives mentioned above. Since TMZ is already an FDA-approved drug, clinical trials on critical cases can be started with the necessary special approvals.

### Materials And Methods

### Cell lines and culture

Cells from patients with VLCAD deficiency FB834 was purchased from Coriell Institute (Cat#GM11408). Others tested including FB671 and FB833, were obtained from patients. The patient had recurrent rhabdomyolysis episodes every 4 weeks and is treated with IV fluids during these episodes. The patient skin biopsies for fibroblast culture were performed on a clinical basis with written informed consent from patients and/or parents. Fibroblasts were cultured in Dulbecco's Modified Eagle Medium (DMEM) supplemented with 15% fetal bovine serum and 100 μg/ml penicillin/streptomycin, 4.5 g/l glucose, 4 mM glutamine and 2 mM pyruvate. Molecular analysis of fibroblast cells FB671 revealed the patient is heterozygote for a VLCAD L540P variant on one allele and has a 9 bases insertion resulting in the additional Asp-Gly-Ala three amino acid insertion at aa 530 (precursor numbering). Molecular analysis of fibroblast cells FB833 revealed heterozygote for two missense mutations causing V174M and E609K replacements. Other fibroblasts used are FB831 homozygous for the MCAD K304E common (>90% of MCAD case) variant, which was also obtained from Coriell Institute (Cat#GM13275), and FB830 homozygous for the HADHA homozygous for the c.1528G>C (p.E510Q, or E474Q with respect to the mature HADHA protein) mutation, Coriell Institute (Cat#GM20266). FB842 is a fibroblast cell line obtained from a patient with LCKAT deficiency, with an S70P amino acid residue replacement on HADHB allele of TFP and a splicing abnormality in the other allele.

Trimetazidine, 1-(2,3,4-trimethoxybenzyl)piperazine, was obtained from Sigma-Aldrich Co., St. Louis, MO. The powder was dissolved in phosphate buffer saline, PBS, as a 200 mM stock solution. Amounts were added appropriately directly to cell culture media in T175 flasks when the cultures were about 85-90 confluent. The cultures were allowed to grow for 48 h at 3TC, and then harvested. Harvested cell pellets were stored at −80° C. until immune and enzymatic assays analyses. One to 1.5 ml media samples were also stored at −80° C. for acylcarnitines.

### Confocal Imaging and Immunofluorescence Intensity Quantification of Fibroblasts

Fibroblasts from VLCAD deficient patient that were untreated and treated with varying concentrations of TMZ were seeded at a concentration of 5×10⁴ cells/ml on tissue culture-treated glass cover slips and allowed to grow overnight with various TMZ concentrations included at 37° C. in a 5% CO₂, 95% humidity incubator. Cells were then fixed in 4% paraformaldehyde for 10 min followed by 0.1% Triton X100 cell permeabilization and further blocked after brief washings in 5% donkey serum (Jackson ImmunoResearch, WestGrove, PA) for 1 hour on ice. This was followed by double primary antibody incubation with anti-VLCAD antibody, anti-TFP, or anti MCAD generated by Cocalico Biologicals, or obtained from Abcam, Cambridge, MA, or Santa Cruz, CA, at 4° C. overnight. After brief washing, cells were further incubated with donkey anti-rabbit secondary antibody Alexa Fluor 488 (Invitrogen, Grand Island, NY) for VLCAD. Nuclei were counterstained with DAPI. The cover slips were then mounted using mounting media before imaging. All the images were taken using a Olympus Confocal FluoView FV1000 microscope at a magnification of 40 and 60×. Immunofluorescence intensity was quantified using NIH ImageJ software.

### ETF Fluorescence Reduction ACAD Activity Assay

This is the standard enzyme assay we use to measure ACAD enzyme activity at the picomoles level in tissues and in cell culture. Assay protocol with the key ingredient being ETF (electron transfer flavoprotein) that is isolated from pig liver has been published (Vockley et al., 2000).

### Level of Expression of VLCAD

The effect of increasing amounts of TMZ on ACADVL gene expression in FB834 cells was monitored using standard qRT-PCR protocol. Messenger RNA transcription levels of ACADVL (MIM: 609575) for the patient's fibroblasts cell lines with VLCAD deficiency untreated and treated with TMZ were quantified via qRT-PCR with an Applied Biosystems StepOnePlus instrument using TaqMan® Gene Expression Master Mix (from ThermoFisher Scientific). The reference sample was fibroblasts with no VLCAD deficiency. Human GAPDH was used as an endogenous control. Commercial primers for ACADVL and GAPDH were used using and TaqMan™ Gene Expression Assay (ThermoFisher Scientific), which consists of a pair of unlabeled PCR primers and a TaqMan probe with a FAM™ or VICO dye label on the 5″-end and minor groove binder (MGB) and non-fluorescent quencher (NFQ) on the 3″-end. The relative quantity RQ of the samples was compared between the reference sample, treated VLCAD deficiency cell lines untreated and treated with TMZ.

### Acylcarnitine Levels Determination

Acylcarnitines level determination was performed at CHOPS, Philadelphia, PA.

### Mitochondrial Superoxide Production

Cell suspension containing 1×10⁵ cells per mL were incubated for 25 min at 37° C. with 150 nM Mitotracker Green (Invitrogen, Grand Island, NY) for mitochondrial mass evaluation or for 15 min at 37° C. with 5 μM Mitosox Red, from Invitrogen, for superoxide production measurement. After incubation, 10,000 cells samples were analyzed in a Becton Dickinson FACSAria II flow cytometer.

### Experimental Evidence

### Protein Presence Changes Detected by Western Blots and Cell Immunostaining Assays

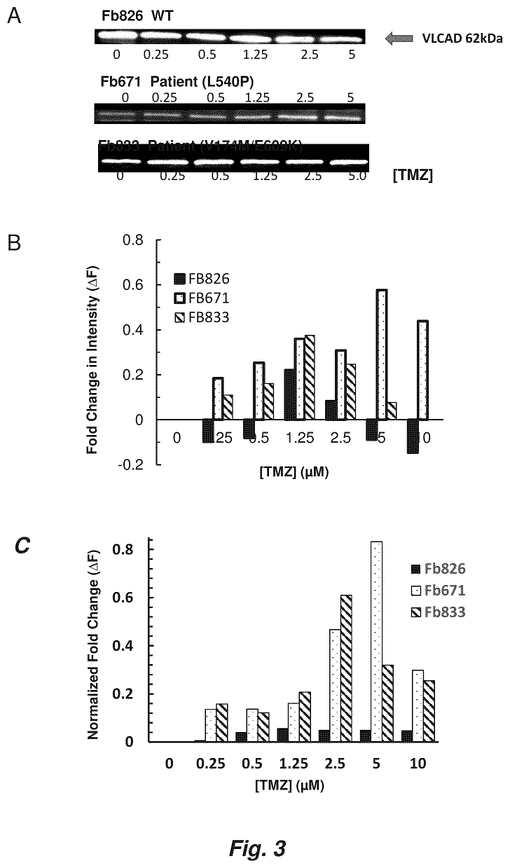

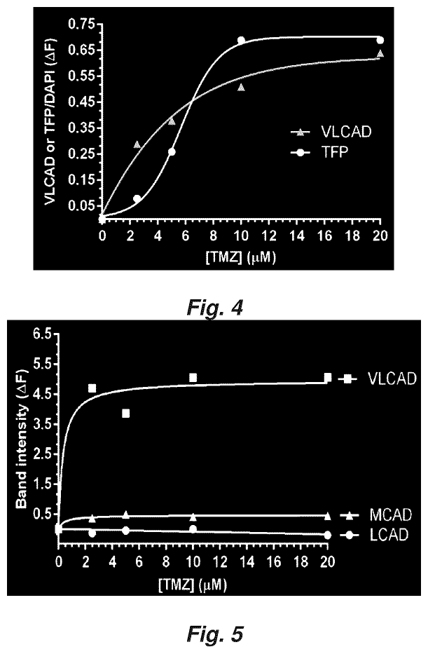

Monitoring the VLCAD presence detected by western blotting in the VLCAD deficient cell line FB833 and FB671 shows proportional increase in VLCAD signal in patients' cells when treated with increased amount of TMZ incubated for 48 hrs, FIGS. 3A and B. Monitoring of VLCAD presence detected by immunostaining in the same VLCAD deficient cell lines shows proportional increase in VLCAD signal in patients' cells when treated with increased amount of TMZ incubated for 72 hrs as well, FIG. 3C. In situ monitoring of VLCAD presence detected by immunostaining in the VLCAD deficient cell line FB834 and similarly it shows proportional increase in VLCAD signal in patients' cells when treated with increased amount of TMZ incubated for 72 hrs, FIG. 4. FIG. 4 shows the fold increase in the total VLCAD and TFP detected using fluorescence (green) normalized to total nuclear DAPI fluorescence (blue). FIG. 5 shows the VLCAD, MCAD, and LCAD signal detected by western blotting. While VLCAD shows more significant response compared to MCAD, this is consistent with published reports on the inhibition of LCKAT, the long chain enzyme, more significantly compared to the medium chain 3-ketoacyl-CoA thiolase. LCAD was unaffected in this cell line, but this may be expected.

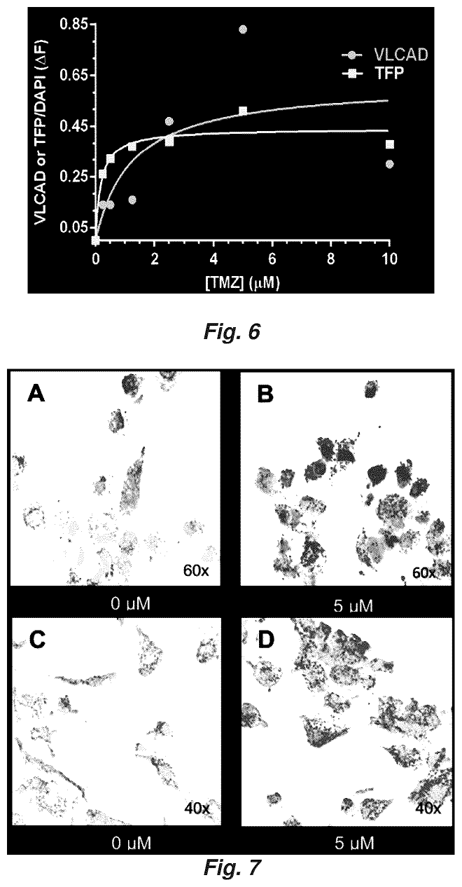

In situ detection of VLCAD presence, detected by immunostaining in the VLCAD deficient cell line FB671 shows proportional increase in VLCAD signal in patients' cells when treated with increased amount of TMZ incubated for 72 hrs, FIG. 6. FIG. 6 shows the fold increase in the total VLCAD green fluorescence normalized to total nuclear DAPI blue fluorescence.

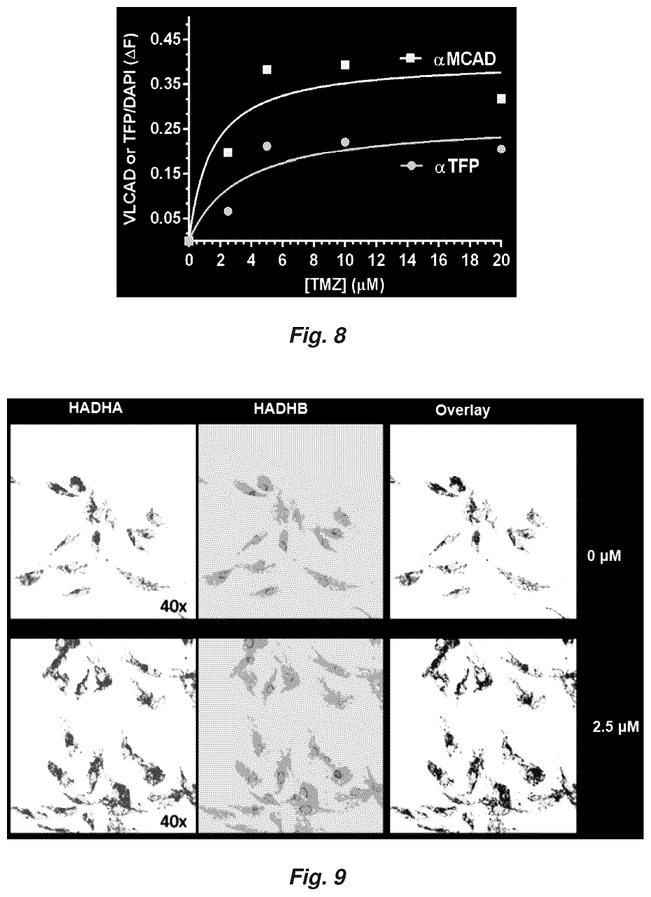

An increase in MCAD antigen signal was observed in the MCAD deficient cell line FB831, increasing from barely detectable to detectable with TMZ concentration of 5 μM. FB831 was also immunostained for TFP presence and increase of antigen was detected peaking at 5 μM of TMZ, (FIGS. 7 and 8).

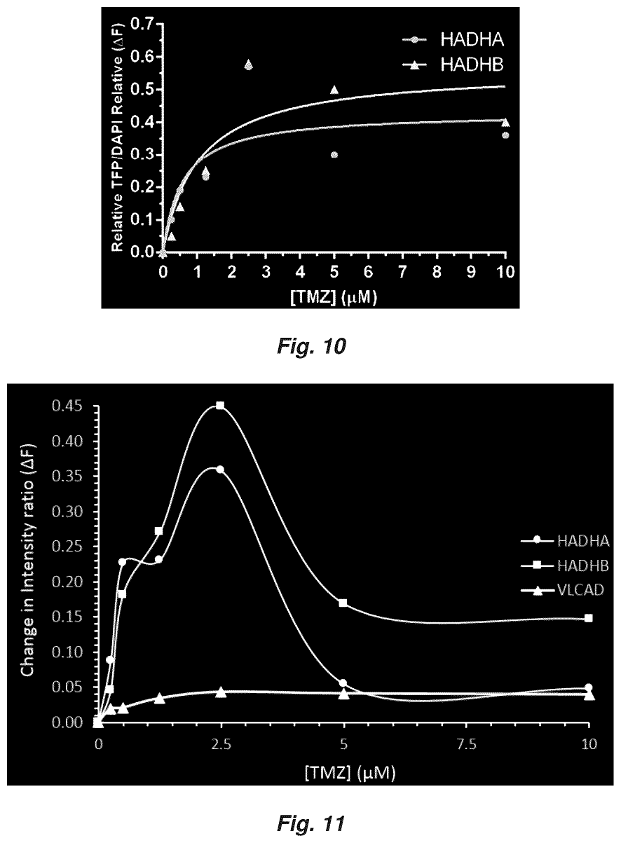

An increase in HADHA and HADHB antigen signal was observed in the LCHAD (part of HADHA subunit of TFP) deficient cell line FB830, increasing maximum at TMZ concentration of 2.5 μM. FB830 was also immunostained, (FIGS. 9 and 10). FIG. 11 shows the effect of the presence of increasing amounts, 0.25, 0.5, 1.25, 2.5, 5.0 and 10 μM, of TMZ in FB842 LCKAT deficient fibroblast cells on HADHA, HADHB, and VLCAD presence.

### VLCAD Enzyme Activity

In this current study, the ETF fluorescence reduction assay was used, which is a superior and more sensitive and reliable assay for measuring ACAD enzyme activity. In two patients' cell lines, FB834 and FB671, VLCAD activity significantly increased proportionally with increasing concentrations of TMZ in the cell culture media incubated for 48 hrs. The fold increase in VLCAD enzyme activity is essentially matching the fold increase in antigen presence as observed by in situ immunostaining, FIG. 4.

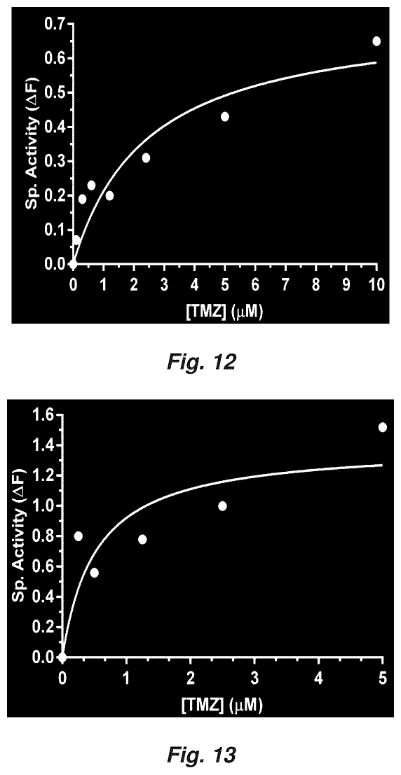

FIGS. 12 and 13 show the relative increase in VLCAD activity at various concentrations of TMZ, in two cell lines from patients with VLCAD deficiency, FB834 and FB671, respectively. In both cases VLCAD enzyme activity increased with increasing amounts of TMZ. The increase varied in magnitude by ˜0.6 and ˜1.3 fold, respectively.

### Level of Expression of VLCAD

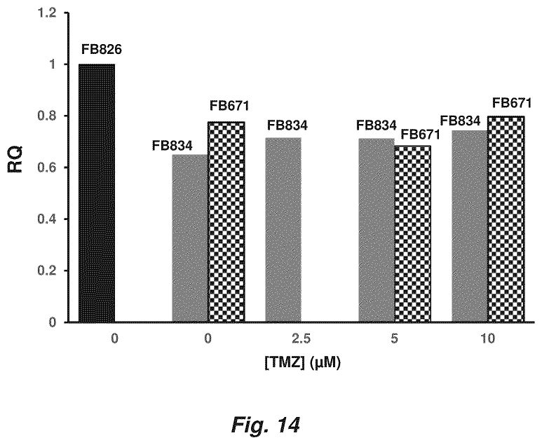

No significant effect was observed on ACADVL expression in FB834 with increasing amounts of TMZ (FIG. 14).

### Acylcarnitine Levels

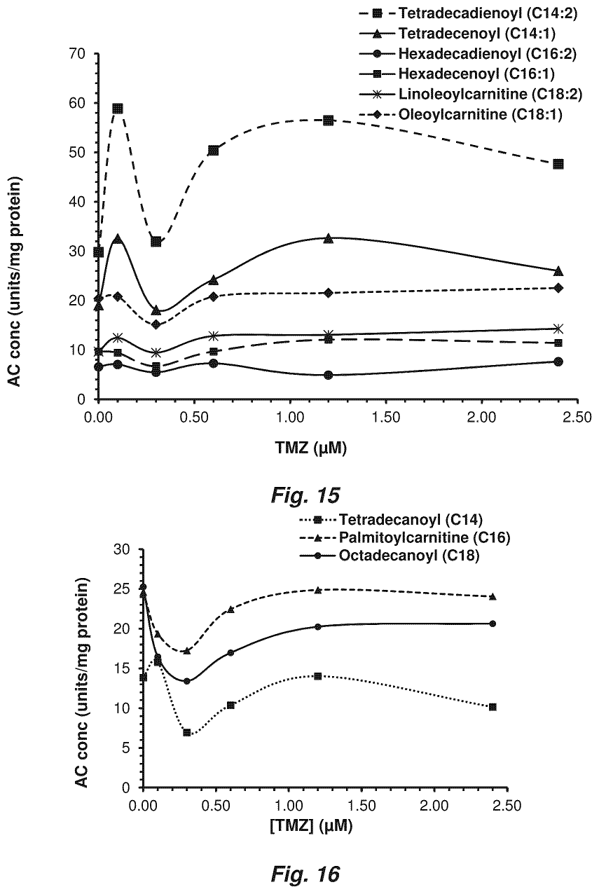

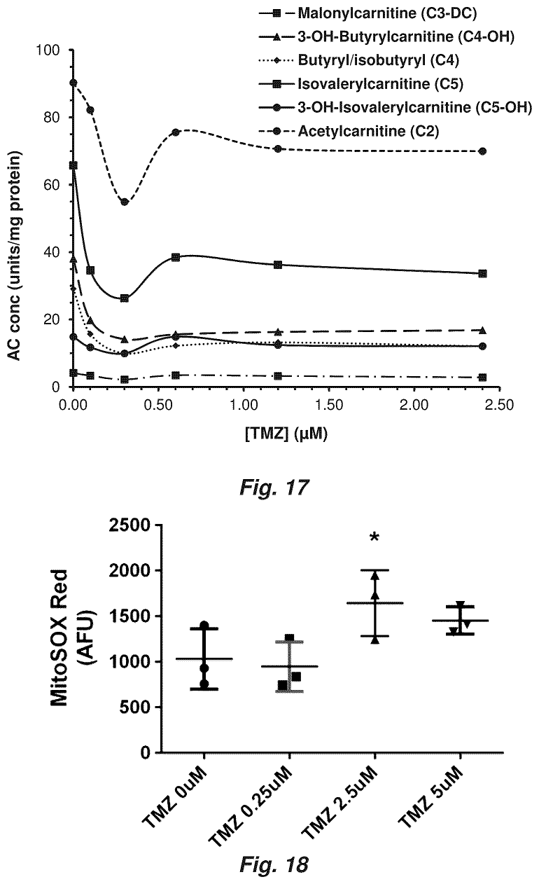

Most of the acylcarnitines, as shown in FIGS. 15-17, were reduced with maximum reduction at ˜0.25 μM level. The percentage drop in each acylcarnitine ranges ˜30-40%. Such a drop would be clinically significant.

### Mitochondrial Superoxide Production

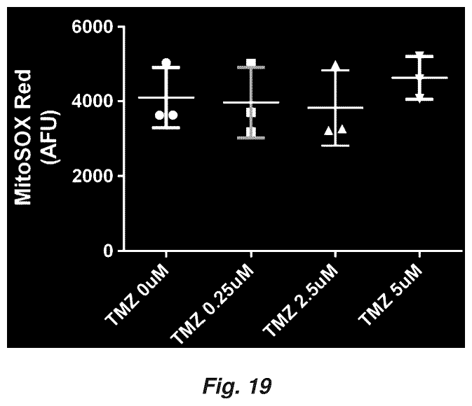

The reactive oxygen species (ROS) has been observed to be higher in fibroblast cells from patients with VLCAD deficiency compared to control cell lines. The mechanism is unknown and the contribution of higher ROS on the pathological phenotype has not been determined. ROS was measured in FB834 and FB671 at various concentrations of TMZ, FIGS. 18 and 19, respectively.

### Conclusion

The efficacy of this drug to improve the parameters of VLCAD viability at the molecular level in deficient cell lines is evident from the above in vitro experimental results. Key end points of validated experimental tests were met, including increase in VLCAD enzyme activity, increase in VLCAD protein presence observed in situ, and in case of VLCAD deficiency lower C16 present in the media at lower amount of TMZ, ˜2.5 μM. The concentration-dependent increase in detected in situ antigen and in enzyme activity measured in vitro was not a function of increased ACADVL gene expression as shown by RT-qPCR data, but rather decreased turnover.

While the unexplained increase in ROS was observed in FB671 and in FB834 VLCAD deficient cell line, the increase in ROS is still not understood, and secondary unknown indirect effects unrelated to the VLCAD deficiency cannot be ruled out. Clinically, the patient, where FB671 was collected, has shown increase in inflammation parameters that could be related to such higher ROS and so other therapeutic interventions could be considered.

The increase in VLCAD enzyme activity was variable among the two patient cell lines tested FB834 and FB671. This is also consistent with VLCAD mutants expected to vary in their stability and consistent with mechanism of action of the drug proposed above. While some missense mutations will lead to completely crippled VLCAD, others that have milder effect on protein stability may respond well to TMZ treatment, and hence patients would be expected to respond differently and it may become necessary to adjust/customize TMZ doses according to the patient. While TMZ is one among others that act as inhibitors of fatty acid β-oxidation, other inhibitors of fatty acid β-oxidation with potentially can induce similar improvements in protein presence.

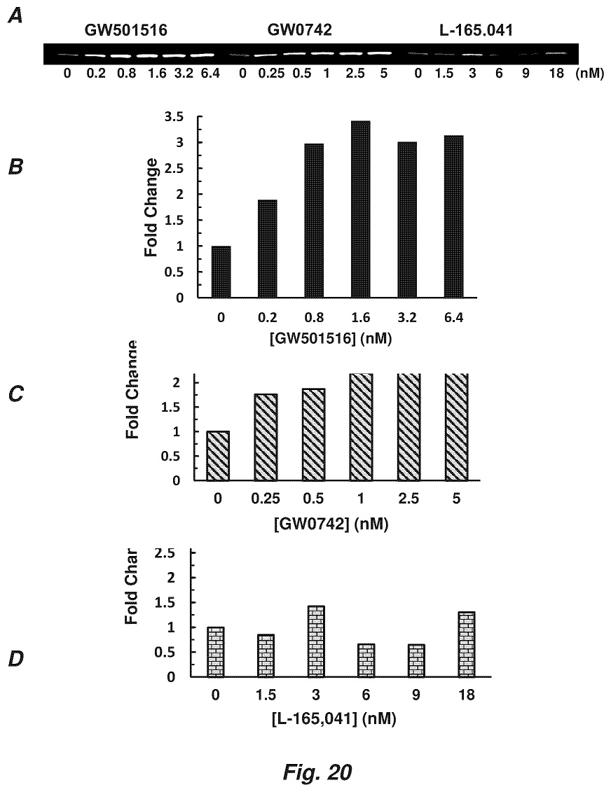

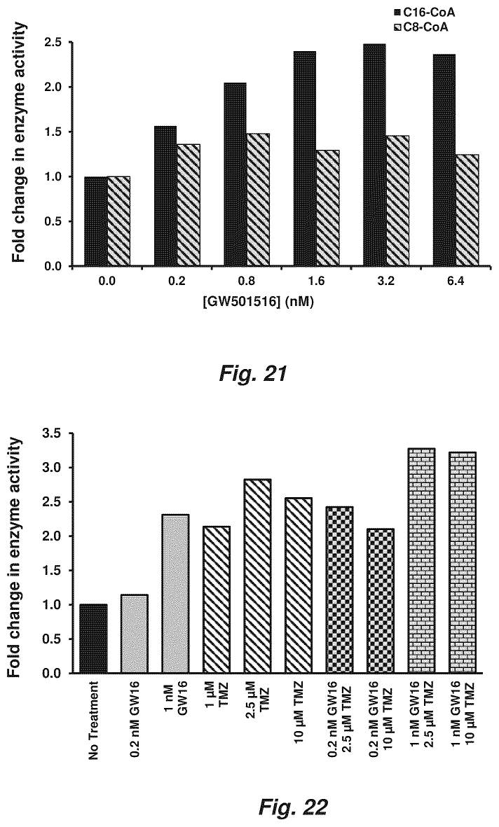

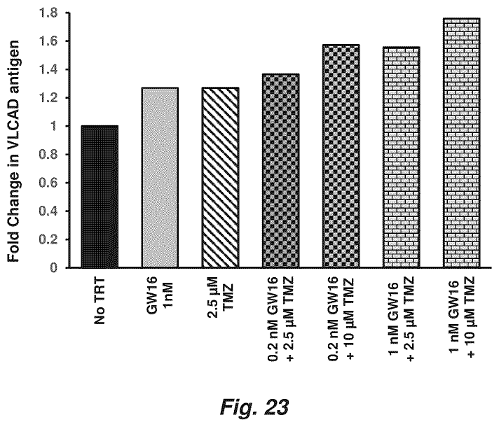

Three PPARδ were tested for efficacy to enhance expression of the ACADVL and ACADM genes. These were GW501516, GW0742 (a.k.a. GW610742), and L-165,0411. While the former two have shown increase in VLCAD antigen signal in FB833, the latter one did not show as much (FIG. 20). Hypothesizing that the mechanism of the PPARδ will work well to complement the effect of trimetazidine, trimmetazidine and GW501516 were tried in combination. Enhancement with both of them present was highest at 10 μM and 1 nM, respectively, see FIGS. 21-23.

The following clauses are illustrative of various aspects of the invention:

Clause 1: A method of treating a disorder caused by a mutation, for example a missense mutation, in any of the enzymes and/or proteins of the β-oxidation pathway and/or fatty acid mitochondrial transport in a patient in need thereof, comprising administering to the patient an effective amount of an inhibitor of activity of an enzyme of the β-oxidation pathway downstream in the β-oxidation pathway of the enzyme having the mutation, thereby increasing activity of the enzyme of the β-oxidation pathway of fatty acid mitochondrial transport having the mutation, wherein the product of the enzyme having the mutation stabilizes that unstable enzyme, or one or more substrates or products of the downstream enzyme binds, for example allosterically, to the mutant enzyme and stabilize that unstable enzyme.

Clause 2: A method of increasing activity of a protein and/or enzyme of the β-oxidation pathway and/or fatty acid mitochondrial transporter protein/enzyme, the protein and/or enzyme having a mutation, for example a missense mutation, resulting in loss of its activity or stability, in a patient in need thereof, comprising administering to the patient an effective amount of an inhibitor of activity of an enzyme of the β-oxidation pathway downstream in the β-oxidation pathway of the enzyme having the mutation, thereby increasing activity of the upstream enzyme of the β-oxidation pathway and/or a fatty acid mitochondrial transporter protein/enzyme having a mutation, wherein the product of the enzyme having the mutation binds back and stabilizes that protein/enzyme.

Clause 3: The method of clause 1 or clause 2, wherein the mutation is a missense mutation, optionally a missense mutation that does not result in a complete loss of enzyme activity, e.g., the mutant enzyme still has some partial activity that is stabilized by the method.

Clause 4: The method of any of clauses 1-3, wherein the mutated enzyme is MCAD.

Clause 5: The method of any of clauses 1-4, wherein the mutation is K304E.

Clause 6: The method of any of clauses 1-5 wherein the mutated enzyme is VLCAD.

Clause 7: The method of any of clauses 1-3 and 6, wherein the mutation is L540P.

Clause 8: The method of any of clauses 1-7, wherein the mutated enzyme is an HADHA or HADHB subunit of TFP.

Clause 9: The method of any of clauses 1-3 and 8, wherein the mutation is E510Q of HADHA.

Clause 10: The method of any of clauses 1-3 and 8, wherein the mutation is R247C of HADHB.

Clause 11: The method of any of clauses 1-10, wherein the enzyme of the β-oxidation pathway downstream in the β-oxidation pathway to the enzyme having the missense mutation is long-chain 3-ketoacyl-CoA thiolase (LCKAT).

Clause 12: The method of any of clauses 1-11, wherein activity of an enzyme of the β-oxidation pathway downstream in the β-oxidation pathway to the enzyme having the missense mutation is inhibited by trimetazidine or a derivative thereof, or a pharmaceutically-acceptable salt or ester thereof.

Clause 13: The method of any of clauses 1-12, wherein the activity of an enzyme of the β-oxidation pathway downstream in the β-oxidation pathway to the enzyme having the missense mutation is inhibited by a trimetazidine derivative, or a pharmaceutically-acceptable salt or ester thereof. Clause 14: The method of clause 13, wherein the trimetazine derivative has the structure:

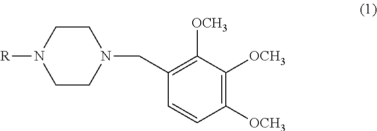

where R is a 5 to 7 member hydrocarbon or heterocyclic group substituted with one or more C₁-C₆ hydrocarbon groups.

Clause 15: The method of clause 14, wherein R is 2,2,5,5-tetramethylpyrrolinyl that is optionally substituted at its 4 position with a C₁-C₆ hydrocarbon group, such as a phenyl group.

Clause 16: The method of clause 14, wherein R is 2,2,5,5-tetramethyl-4-phenylpyrrolinyl.

Clause 17: The method of any of clauses 1-16, wherein the enzyme having the missense mutation is in a mitochondrial membrane fatty acid transport protein.

Clause 18: The method of clause 17, wherein the mitochondrial membrane fatty acid transport protein is carnitine palmitoyltransferase I (CPT I) or carnitine palmitoyltransferase 2 (CPT II).

Clause 19: The method of any of clauses 1-18, further comprising administering to the patient an amount of a Peroxisome Proliferator-Activated Receptor delta (PPARδ) agonist effective to increase activity of the β-oxidation pathway in a patient.

Clause 20: The method of clause 19, wherein the PPARδ agonist is chosen from GW501516, GW0742, or any PPARδ agonist that enhances production of an enzyme of the β-oxidation pathway and/or fatty acid mitochondrial transport having a misense mutation.

Having described this invention, it will be understood to those of ordinary skill in the art that the same can be performed within a wide and equivalent range of conditions, formulations and other parameters without affecting the scope of the invention or any embodiment thereof.

### REFERENCES

Aliefendioglu et al. (2007). A newborn with VLCAD deficiency. Clinical, biochemical, and histopathological findings. Eur J Pediatr 166, 1077-1080.

Ancerewicz et al. (1998). Structure—property relationships of trimetazidine derivatives and model compounds as potential antioxidants. Free Rad. Biol Med 25 (1):113-120.

Andresen et al. (1999). Clear correlation of genotype with disease phenotype in very-long-chain acyl-CoA dehydrogenase deficiency. Am. J. Hum. Genet. 64, 479-494.

Arnold et al. (2009) A Delphi clinical practice protocol for the management of very long chain acyl-CoA dehydrogenase deficiency. Mol Genet Metab 96:85-90.

Boneh et al. (2006). VLCAD deficiency: pitfalls in newborn screening and confirmation of diagnosis by mutation analysis. Mol Genet Metab 88, 166-170.

Goetzman et al. (2007). Expression and characterization of mutations in human very long-chain acyl-CoA dehydrogenase using a prokaryotic system. Mol Genet Metab 91, 138-147.

Goetzman et al. (2014). Long-chain acyl-CoA dehydrogenase deficiency as a cause of pulmonary surfactant dysfunction. J Biol Chem 289, 10668-10679.

Gregersen et al. (2008). Mitochondrial fatty acid oxidation defects—remaining challenges. J Inherit Metab Dis 31, 643-657.

Kálai et al. (2006) Structure—activity studies on the protection of Trimetazidine derivatives modified with nitroxides and their precursors from myocardial ischemia-reperfusion injury. Bioorg Medicinal Chem 14:5510-5516.

Kamijo et al. (1994) Mitochondrial trifunctional protein deficiency—catalytic heterogeneity of the mutant enzyme in two patients. J Clin Invest 93, 1740-1747.

Kantor et al. (2000). The antianginal drug trimetazidine shifts cardiac energy metabolism from fatty acid oxidation to glucose oxidation by inhibiting mitochondrial long-chain 3-ketoacyl coenzyme A thiolase. Circ Res 86, 580-588.

Kutala et al. (2006) Attenuation of myocardial ischemia-reperfusion injury by trimetazidine derivatives functionalized with antioxidant properties. J Pharmacol Exper Therapeut 317 (3):921-928.

Laforet et al. (2009). Diagnostic assessment and long-term follow-up of 13 patients with Very Long-Chain Acyl-Coenzyme A dehydrogenase (VLCAD) deficiency. Neuromuscul Disord 19, 324-329.

Lehman et al. (1990). An acyl-coenzyme A dehydrogenase assay utilizing the ferricenium ion. Analytic. Biochem. 186, 280-284.

Mathur, et al. (1999). Molecular heterogeneity in very-long-chain acyl-CoA dehydrogenase deficiency causing pediatric cardiomyopathy and sudden death. Circulation 99, 1337-1343.

Mohsen et al. (1995). Ligand-induced conformational changes of thymidylate synthase detected by limited proteolysis. Biochemistry, 34:1669-77.

Nasser et al. (2004). Thermal unfolding of medium-chain acyl-CoA dehydrogenase and iso(3)valeryl-CoA dehydrogenase: study of the effect of genetic defects on enzyme stability. Biochim Biophys Acta 1690, 22-32.

Pena et al. (2016). Outcomes and genotype-phenotype correlations in 52 individuals with VLCAD deficiency diagnosed by NBS and enrolled in the IBEM-IS database. Mol Genet Metab.

Pons et al. (2000). Clinical and molecular heterogeneity in very-long-chain acyl-coenzyme A dehydrogenase deficiency. Pediatr Neurol 22, 98-105.

Spiekerkoetter et al. (2009) Management and outcome in 75 individuals with long-chain fatty acid oxidation defects: results from a workshop. J Inherit Metab Dis 32:488-497.

Spiekerkoetter et al. (2004) The early-onset phenotype of mitochondrial trifunctional protein deficiency: A lethal disorder with multiple tissue involvement. Journal of Inherited Metabolic Disease 27, 294-296.

Spiekerkoetter et al. (2003) Molecular and phenotypic heterogeneity in mitochondrial trifunctional protein deficiency due to beta-subunit mutations. Hum Mutat 21, 598-607.

Stanley et al. (2003). Metabolic therapy in the treatment of ischaemic heart disease: the pharmacology of trimetazidine. Fundam Clin Pharmacol 17, 133-145.

Tang (2006). Metabolic approach in heart failure: rethinking how we translate from theory to clinical practice. J Am Coll Cardiol 48, 999-1000.

Vockley et al. (2000). Mammalian branched-chain acyl-CoA dehydrogenases: molecular cloning and characterization of recombinant enzymes. Methods Enzymol 324, 241-258.

Watanabe et al. (2000). Molecular basis of very long chain acyl-CoA dehydrogenase deficiency in three Israeli patients: identification of a complex mutant allele with P65L and K247Q mutations, the former being an exonic mutation causing exon 3 skipping. Hum Mutat 15, 430-438.

Zhang et al. (2014). Clinical features and mutations in seven Chinese patients with very long chain acyl-CoA dehydrogenase deficiency. World J Pediatr 10, 119-125.

We claim:

1. A method of increasing activity of an upstream enzyme in a patient in need thereof, the upstream enzyme comprising a fatty acid β-oxidation pathway or fatty acid mitochondrial transporter protein/enzyme having a missense mutation and selected from the group consisting of very long chain acyl-CoA dehydrogenase (VLCAD), medium chain acyl-CoA dehydrogenase (MCAD), mitochondrial trifunctional protein (TFP), long-chain 3-ketoacyl-CoA thiolase (LCKAT), and mitochondrial membrane fatty acid transport protein, the method comprising:
administering, to the patient, an effective amount of an inhibitor of activity of an enzyme of the β-oxidation pathway downstream in the β-oxidation pathway of the upstream enzyme, thereby increasing activity of the upstream enzyme, wherein the product of the upstream enzyme stabilizes the upstream enzyme.

2. The method of claim 1, wherein the missense mutation is K304E of MCAD, or any other MCAD harboring a homozygous or heterozygous missense mutation.

3. The method of claim 1, wherein the activity of the enzyme of the β-oxidation pathway downstream in the β-oxidation pathway to the upstream enzyme is inhibited by a trimetazidine, or a pharmaceutically-acceptable salt or ester thereof.

4. The method of claim 1, wherein the activity of the enzyme of the β-oxidation pathway downstream in the β-oxidation pathway to the upstream enzyme is inhibited by a trimetazidine derivative, or a pharmaceutically-acceptable salt or ester thereof. structure:

5. The method of claim 4, wherein the trimetazidine derivative has the structure: 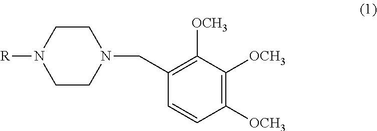
where R is a 5 to 7 member hydrocarbon or heterocyclic group substituted with one or more C₁-C₆ hydrocarbon groups.

6. The method of claim 5, wherein R is 2,2,5,5-tetramethylpyrrolinyl that is optionally substituted at its 4 position with a C₁-C₆ hydrocarbon group, such as a phenyl group.

7. The method of claim 5, wherein R is 2,2,5,5-tetramethyl-4-phenylpyrrolinyl.

8. The method of claim 1, wherein the missense mutation does not result in a complete loss of enzyme presence or activity.

9. The method of claim 1, wherein the missense mutation is L540P, V174M, E609K, or any other VLCAD or VLCAD isoforms, harboring any destabilizing missense mutation(s).

10. The method of claim 1, wherein the TFP is the α-subunit or the β-subunit encoded by HADHA or HADHB genes, respectively.

11. The method of claim 10, wherein the missense mutation is E510Q of HADHA or R247C of HADHB, or any destabilizing missense mutations thereof.

12. The method of claim 1, wherein the enzyme of the β-oxidation pathway downstream in the β-oxidation pathway of the upstream enzyme is long/medium-chain 3-ketoacyl-CoA thiolase (LCKAT) subunit.

13. The method of claim 1, wherein the upstream enzyme is a mitochondrial membrane fatty acid transport protein selected from carnitine palmitoyltransferase I (CPT I) and carnitine palmitoyltransferase 2 (CPT II).

14. The method of claim 1, further comprising administering to the patient an amount of a Peroxisome Proliferator-Activated Receptor delta (PPARδ) agonist effective to increase production and activity of the upstream enzyme in the patient.

15. The method of claim 14, wherein the PPARδ agonist is chosen from GW501516 and GW0742.

16. The method of claim 1, wherein the patient is experiencing MCAD deficiency.


SEQUENCE LISTING (6 sequences)

SEQ ID NO: 1 - protein, 655 aa, Homo sapiens
MQAARMAASLGRQLLRLGGGSSRLTALLGQPRPGPARRPYAGGAAQLALDKSDSHPSDALTRKKPAKAESKSFAVGMFKGQLTTDQVFPYPSVLNEEQTQFLKELVEPVSRFFEEVNDPAKNDALEMVEETTWQGLKELGAFGLQVPSELGGVGLCNTQYARLVEIVGMHDLGVGITLGAHQSIGFKGILLFGTKAQKEKYLPKLASGETVAAFCLTEPSSGSDAASIRTSAVPSPCGKYYTLNGSKLWISNGGLADIFTVFAKTPVTDPATGAVKEKITAFVVERGFGGITHGPPEKKMGIKASNTAEVFFDGVRVPSENVLGEVGSGFKVAMHILNNGRFGMAAALAGTMRGIIAKAVDHATNRTQFGEKIHNFGLIQEKLARMVMLQYVTESMAYMVSANMDQGATDFQIEAAISKIFGSEAAWKVTDECIQIMGGMGFMKEPGVERVLRDLRIFRIFEGTNDILRLFVALQGCMDKGKELSGLGSALKNPFGNAGLLLGEAGKQLRRRAGLGSGLSLSGLVHPELSRSGELAVRALEQFATVVEAKLIKHKKGIVNEQFLLQRLADGAIDLYAMVVVLSRASRSLSEGHPTAQHEKMLCDTWCIEAAARIREGMAALQSDPWQQELYRNFKSISKALVERGGVVTSNPLGF

SEQ ID NO: 2 - protein, 430 aa, Homo sapiens [sequence omitted]

SEQ ID NO: 3 - protein, 421 aa, Homo sapiens
MAAGFGRCCRVLRSISRFHWRSQHTKANRQREPGLGFSFEFTEQQKEFQATARKFAREEIIPVAAEYDKTGEYPVPLIRRAWELGLMNTHIPENCGGLGLGTFDACLISEELAYGCTGVQTAIEGNSLGQMPIIIAGNDQQKKKYLGRMTEEPLMCAYCVTEPGAGSDVAGIKTKAEKKGDEYIINGQKMWITNGGKANWYFLLARSDPDPKAPANKAFTGFIVEADTPGIQIGRKELNMGQRCSDTRGIVFEDVKVPKENVLIGDGAGFKVAMGAFDKTRPVVAAGAVGLAQRALDEATKYALERKTFGKLLVEHQAISFMLAEMAMKVELARMSYQRAAWEVDSGRRNTYYASIAKAFAGDIANQLATDAVQILGGNGFNTEYPVEKLMRDAKIYQIYEGTSQIQRLIVAREHIDKYKN

SEQ ID NO: 4 - protein, 412 aa, Homo sapiens
MAAALLARASGPARRALCPRAWRQLHTIYQSVELPETHQMLLQTCRDFAEKELFPIAAQVDKEHLFPAAQVKKMGGLGLLAMDVPEELGGAGLDYLAYAIAMEEISRGCASTGVIMSVNNSLYLGPILKFGSKEQKQAWVTPFTSGDKIGCFALSEPGNGSDAGAASTTARAEGDSWVLNGTKAWITNAWEASAAVVFASTDRALQNKGISAFLVPMPTPGLTLGKKEDKLGIRGSSTANLIFEDCRIPKDSILGEPGMGFKIAMQTLDMGRIGIASQALGIAQTALDCAVNYAENRMAFGAPLTKLQVIQFKLADMALALESARLLTWRAAMLKDNKKPFIKEAAMAKLAASEAATAISHQAIQILGGMGYVTEMPAERHYRDARITEIYEGTSEIQRLVIAGHLLRSYRS

SEQ ID NO: 5 - protein, 763 aa, Homo sapiens
MVACRAIGILSRFSAFRILRSRGYICRNFTGSSALLTRTHINYGVKGDVAVVRINSPNSKVNTLSKELHSEFSEVMNEIWASDQIRSAVLISSKPGCFIAGADINMLAACKTLQEVTQLSQEAQRIVEKLEKSTKPIVAAINGSCLGGGLEVAISCQYRIATKDRKTVLGTPEVLLGALPGAGGTQRLPKMVGVPAALDMMLTGRSIRADRAKKMGLVDQLVEPLGPGLKPPEERTIEYLEEVAITFAKGLADKKISPKRDKGLVEKLTAYAMTIPFVRQQVYKKVEEKVRKQTKGLYPAPLKIIDVVKTGIEQGSDAGYLCESQKFGELVMTKESKALMGLYHGQVLCKKNKFGAPQKDVKHLAILGAGLMGAGIAQVSVDKGLKTILKDATLTALDRGQQQVFKGLNDKVKKKALTSFERDSIFSNLTGQLDYQGFEKADMVIEAVFEDLSLKHRVLKEVEAVIPDHCIFASNTSALPISEIAAVSKRPEKVIGMHYFSPVDKMQLLEIITTEKTSKDTSASAVAVGLKQGKVIIVVKDGPGFYTTRCLAPMMSEVIRILQEGVDPKKLDSLTTSFGFPVGAATLVDEVGVDVAKHVAEDLGKVFGERFGGGNPELLTQMVSKGFLGRKSGKGFYIYQEGVKRKDLNSDMDSILASLKLPPKSEVSSDEDIQFRLVTRFVNEAVMCLQEGILATPAEGDIGAVFGLGFPPCLGGPFRFVDLYGAQKIVDRLKKYEAAYGKQFTPCQLLADHANSPNKKFYQ

SEQ ID NO: 6 - protein, 474 aa, Homo sapiens
MTILTYPFKNLPTASKWALRFSIRPLSCSSQLRAAPAVQTKTKKTLAKPNIRNVVVVDGVRTPFLLSGTSYKDLMPHDLARAALTGLLHRTSVPKEVVDYIIFGTVIQEVKTSNVAREAALGAGFSDKTPAHTVTMACISANQAMTTGVGLIASGQCDVIVAGGVELMSDVPIRHSRKMRKLMLDLNKAKSMGQRLSLISKFRFNFLAPELPAVSEFSTSETMGHSADRLAAAFAVSRLEQDEYALRSHSLAKKAQDEGLLSDVVPFKVPGKDTVTKDNGIRPSSLEQMAKLKPAFIKPYGTVTAANSSFLTDGASAMLIMAEEKALAMGYKPKAYLRDFMYVSQDPKDQLLLGPTYATPKVLEKAGLTMNDIDAFEFHEAFSGQILANFKAMDSDWFAENYMGRKTKVGLPPLEKFNNWGGSLSLGHPFGATGCRLVMAAANRLRKEGGQYGLVAACAAGGQGHAMIVEAYPK